(19) United States
(12) Patent Application Publication
(10) Pub. No.: US 2024/0382288 A1
(43) Pub. Date: Nov. 21, 2024

(54) SYSTEMS, DEVICES, AND METHODS FOR TOOTH POSITIONING

(71) Applicant: MODJAW, Villeurbanne (FR)

(72) Inventors: Maxime Jaisson, Porte-de-Savoie (FR); Antoine Jules Rodrigue, Lyon (FR)

(21) Appl. No.: 18/692,617

(22) PCT Filed: Sep. 15, 2022
(86) PCT No.: PCT/IB2022/000540

§ 371 Date: Mar. 15, 2024

Related U.S. Application Data
(60) Provisional application No. 63/364,102, filed on May 3, 2022.
(60) Provisional application No. 63/245,072, filed on Sep. 16, 2021.

(51) Int. Cl. A61C 7/00; G16H 50/20

(52) U.S. Cl. CPC A61C 7/002; G16H 50/20

(57) ABSTRACT

This disclosure relates to systems and methods for determining a dental treatment plan. Some embodiments relate to determining a double helix structure. Some embodiments relate to optimizing the positioning of natural teeth, prosthetic teeth, or both to achieve desirable functional and aesthetic characteristics. In some embodiments, machine learning models can be used in determining a treatment plan for a dental patient.

### INCORPORATION BY REFERENCE TO ANY PRIORITY APPLICATIONS

This application claims the benefit of U.S. Provisional Application No. 63/245,072, filed Sep. 16, 2021, and U.S. Provisional Application No. 63/364,102, filed May 3, 2022, and the entirety of these applications is hereby incorporated by reference herein for all purposes.

### BACKGROUND

### Field

The present application relates to systems, devices, and methods for determining, generating, and/or assisting with the tooth positioning for a patient.

### Description of Related Art

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Proper placement of a patient's own teeth, artificial teeth, or both can be important for both aesthetic and functional reasons. Current approaches often fail to consider important information when determining placement of teeth, which can lead to poor aesthetic and/or functional outcomes.

### SUMMARY

The systems, methods, and devices described herein each have several aspects, no single one of which is solely responsible for its desirable attributes. Without limiting the scope of this disclosure, several non-limiting features will now be described briefly.

In some aspects, the techniques described herein relate to a computer-implemented method for dental treatment planning including: receiving, by a computing system, patient data associated with a patient; determining, by the computing system, at least one arc, the arc corresponding to anatomical points of teeth of a tooth library; determining, by the computing system based on the at least one arc, a double helix, the double helix to be used for fitting a tooth library; determining, by the computing system, positions of the teeth of the tooth library on the double helix; and optimizing, by the computing system, the teeth of the tooth library.

In some aspects, the techniques described herein relate to a method, wherein the patient data includes tooth data.

In some aspects, the techniques described herein relate to a method, wherein the patient data includes morphometric data.

In some aspects, the techniques described herein relate to a method, wherein determining at least one arc includes: providing the patient data to an AI model, the AI model trained to identify anatomical points of the teeth of the tooth library.

In some aspects, the techniques described herein relate to a method, wherein determining a double helix includes providing the at least one arc to an AI model configured to determine the double helix based at least in part on the at least one arc.

In some aspects, the techniques described herein relate to a method, wherein optimizing the teeth of the tooth library includes determining, using an AI model, a position, a rotation, or both of each tooth of the tooth library, the AI model configured to optimize functional and aesthetic positioning of the teeth.

In some aspects, the techniques described herein relate to a method, further including performing, by the computing system, dynamic evaluation of the positions of the teeth of the tooth library.

In some aspects, the techniques described herein relate to a method, wherein determining at least one arc includes determining an aesthetic arc, and wherein determining the aesthetic arc includes: projecting, by the computing system, one or more control points onto an image of the patient; defining, by the computing system, based at least in part on the one or more control points, an initial curve; determining a final curve by modifying, by the computing system, at least one control point; and determining, by the computing system, locations of one or more anatomical points based at least in part on the final curve, the locations of the one or more anatomical points defining at least in part the aesthetic arc.

In some aspects, the techniques described herein relate to a method, wherein determining the at least one arc includes determining an aesthetic arc, a centering arc, and a fitting arc.

In some aspects, the techniques described herein relate to a method, wherein determining the least one arc further includes determining a guiding arc associated with mandibular teeth.

In some aspects, the techniques described herein relate to a method, wherein optimizing the teeth of the tooth library includes adjusting a relative positioning of one or more teeth in the tooth library.

In some aspects, the techniques described herein relate to a method, wherein adjusting the relative positioning includes adjusting an overbite value and an overjet value.

In some aspects, the techniques described herein relate to a method, wherein optimizing the teeth of the tooth library includes adjusting any combination of one or more of a size, shape, or rotation of at least one tooth of the tooth library.

In some aspects, the techniques described herein relate to a method, wherein the tooth library includes a library of the patient's teeth, and wherein the method further includes: identifying, by the computing system, one or more teeth of the tooth library; and annotating, by the computing system, one or more anatomical points of each tooth of the one or more teeth of the tooth library.

In some aspects, the techniques described herein relate to a method, wherein the tooth library includes a library of artificial teeth, and wherein the method further includes: selecting, by the computing system based at least in part on the captured patient data, a tooth library from a plurality of prosthetic tooth libraries.

In some aspects, the techniques described herein relate to a method, wherein optimizing the teeth of the tooth library includes determining contact points between maxillary teeth of the patient and mandibular teeth of the patient, wherein performing dynamic evaluation includes determining contact relations between the maxillary teeth of the patient and the mandibular teeth of the patient during movement of a jaw of the patient.

In some aspects, the techniques described herein relate to a system for dental treatment planning including: a computer readable storage medium having program instructions embodied therewith; and one or more processors configured to execute the program instructions to cause the system to: receive patient data associated with a patient; determine at least one arc, the arc corresponding to anatomical points of teeth of a tooth library; determine, based on the at least one arc, a double helix, the double helix to be used for fitting a tooth library; determine positions of the teeth of the tooth library on the double helix; and optimize the teeth of the tooth library.

In some aspects, the techniques described herein relate to a system, wherein the patient data includes tooth data.

In some aspects, the techniques described herein relate to a system, wherein the patient data includes morphometric data.

In some aspects, the techniques described herein relate to a system, wherein determining at least one arc includes: providing the patient data to an AI model, the AI model trained to identify anatomical points of the teeth of the tooth library.

In some aspects, the techniques described herein relate to a system, wherein determining a double helix includes providing the at least one arc to an AI model configured to determine the double helix based at least in part on the at least one arc.

In some aspects, the techniques described herein relate to a system, wherein optimizing the teeth of the tooth library includes determining, using an AI model, a position, a rotation, or both of each tooth of the tooth library, the AI model configured to optimize functional and aesthetic positioning of the teeth.

In some aspects, the techniques described herein relate to a system, wherein the computer readable storage medium has instructions embodied therewith that, when executed by the one or more processors, cause the system to: perform dynamic evaluation of the positions of the teeth of the tooth library.

In some aspects, the techniques described herein relate to a system, wherein determining at least one arc includes determining an aesthetic arc, and wherein determining the aesthetic arc includes: project one or more control points onto an image of the patient; define based at least in part on the one or more control points, an initial curve; define a final curve by modifying at least one control point of the one or more control points; and determine locations of one or more anatomical points based at least in part on the final curve, the locations of the one or more anatomical points defining at least in part the aesthetic arc.

In some aspects, the techniques described herein relate to a system, wherein determining the at least one arc includes determining an aesthetic arc, a centering arc, and a fitting arc.

In some aspects, the techniques described herein relate to a system, wherein determining the at least one arc further including determining a guiding arc associated with mandibular teeth.

In some aspects, the techniques described herein relate to a system, wherein optimizing the teeth of the tooth library includes adjusting a relative positioning of one or more teeth in the tooth library.

In some aspects, the techniques described herein relate to a system, wherein adjusting the relative positioning includes adjusting an overbite value and an overjet value.

In some aspects, the techniques described herein relate to a system, wherein optimizing the teeth of the tooth library includes adjusting any combination of one or more of a size, shape, or rotation of at least one tooth of the tooth library.

In some aspects, the techniques described herein relate to a system, wherein the tooth library includes a library of the patient's teeth, and wherein the computer readable storage medium has instructions embodied therewith that, when executed by the one or more processors, cause the system to: identify one or more teeth of the tooth library; and annotate one or more anatomical points of each tooth of the one or more teeth of the tooth library.

In some aspects, the techniques described herein relate to a system, wherein the tooth library includes a library of artificial teeth, and wherein the computer readable storage medium has instructions embodied therewith that, when executed by the one or more processors, cause the system to: select, based at least in part on the patient data, a tooth library from a plurality of prosthetic tooth libraries.

In some aspects, the techniques described herein relate to a system, wherein optimizing the teeth of the tooth library includes determining contact points between maxillary teeth of the patient and mandibular teeth of the patient, wherein performing dynamic evaluation includes determining contact relations between the maxillary teeth of the patient and the mandibular teeth of the patient during movement of a jaw of the patient.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 shows an example process for generating a tooth positioning plan for a patient according to some embodiments.

FIGS. 2A-21 illustrate an example implementation of an orthodontic process according to some embodiments.

FIGS. 3A-3K illustrate an example implementation of a prosthetic process according to some embodiments.

FIG. 4 illustrates example tooth profiles that can be used for segmenting teeth according to some embodiments.

FIG. 5 illustrates an example for training a machine learning model to carry out some embodiments described herein.

FIG. 6 illustrates annotation of the maxillary incisors 11, 12, 21, and 22 according to some embodiments.

FIG. 7 illustrates annotation of the maxillary canines 13 and 23 according to some embodiments.

FIG. 8 illustrates annotation of the maxillary premolars 14, 15, 24, and 25 according to some embodiments.

FIG. 9 illustrates annotation of the maxillary molars 16, 17, 26, and 27 according to some embodiments.

FIG. 10 illustrates annotation of the mandibular incisors 31, 32, 41, and 42 according to some embodiments.

FIG. 11 illustrates annotation of the mandibular premolars 34 and 44 according to some embodiments.

FIG. 12 illustrates annotation of the mandibular premolars 35 and 45 according to some embodiments.

FIG. 13 illustrates annotation of the mandibular molars 36 and 46 according to some embodiments.

FIG. 14 illustrates annotation of the mandibular molars 37 and 47 according to some embodiments.

FIG. 15 illustrates an example process for determining tooth positioning according to some embodiments.

FIG. 16 illustrates an embodiment of an aesthetic arc that can be manipulated using control points.

FIG. 17 is a block diagram that illustrates an example process for defining a 3D curve according to some embodiments.

FIG. 18 depicts an example of mapping anatomical points from a 2D projection to a 3D capture of a patient's teeth according to some embodiments.

FIG. 19 illustrates an example of anatomical points to which an initial curve has been fitted according to some embodiments.

FIG. 20 illustrates an example process for positioning a tooth library according to some embodiments.

FIG. 21 illustrates an example process for optimizing the static positioning of a patient's teeth according to some embodiments.

FIG. 22 shows an example of points for defining an aesthetic arc for maxillary teeth according to some embodiments.

FIG. 23 shows an example of points defining a centering arc for maxillary teeth according to some embodiments.

FIG. 24 shows an example of points defining a fitting arc for maxillary teeth according to some embodiments.

FIG. 25 shows an example of points defining an aesthetic arc, centering arc, and fitting arc for maxillary teeth according to some embodiments.

FIG. 26 shows an example of points defining a fitting arc for mandibular teeth according to some embodiments.

FIG. 27 shows an example of points defining a centering arc for mandibular teeth according to some embodiments.

FIG. 28 shows an example of points defining a guiding arc for mandibular teeth according to some embodiments.

FIGS. 29-33B illustrate example angular and spatial relationships between teeth and a reference plane.

FIG. 34 shows an example of overbite and overjet.

FIGS. 35A-C illustrate example relationships between overbite, overjet, and various arcs.

FIG. 36 illustrates an example process for developing a treatment plan for a patient.

FIG. 37 illustrates an example computer system that can be used to carry out one or more embodiments disclosed herein.

### DETAILED DESCRIPTION OF THE PREFERRED EMBODIMENT

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of some specific embodiments of the inventions. In addition, embodiments of the inventions can comprise several novel features and no single feature is solely responsible for its desirable attributes or is essential to practicing the inventions herein described.

Unless otherwise noted, the dental notation (e.g., numbering of teeth) used herein conforms to the FDI World Dental Federation notation system (ISO 3950).

### Overview

As discussed briefly above, proper placement of a patient's own teeth, artificial teeth, or both can be important for both aesthetic and functional reasons. Various embodiments described herein relate to systems, methods, and devices for determining, generating, and/or assisting with the tooth positioning of a patient. In some embodiments, the systems, methods, and devices herein can be used for determining, generating, and/or assisting with tooth shaping and/or sizing.

Often, when a practitioner is planning or performing an orthodontic or prosthetic procedure, the practitioner may lack information that would be helpful for positioning teeth, selecting appropriate artificial teeth, and so forth. For example, the practitioner may lack information about the movement of the patient's jaw and/or other morphometric parameters, such as the location of a reference plane (e.g., an axio-orbital plane), which can make it difficult to consider functions such as chewing when determining the placement of a tooth or prosthesis. For example, practitioners may rely on limited static views (e.g., x-rays, cone beam computed tomography (CBCT) scans, and so forth), which can result in the practitioner failing to account for the patient's overall oral and/or facial structure, which can lead to time-consuming procedures (and possibility additional procedures) and/or poor results. For example, with limited information at hand, practitioners may lose sight of the overall architecture of a patient and instead focus on, for example, the positioning and/or arrangement of individual teeth. In some embodiments, morphometric parameters can be unique to a patient. In some embodiments, morphometric parameters can be partially of fully standardized, for example to use a standard axio-orbital plane.

In some cases, practitioners may develop a treatment plan that focuses on aesthetics. While such an approach can deliver a pleasing aesthetic result, it may result in functional problems. For example, a patient may experience premature wearing (e.g., due to erosion or abrasion) of tooth surfaces, increased vulnerability to cracking or chipping, difficulty eating or speaking, and so forth, if functional aspects of a patient's teeth are neglected. In some cases, practitioners may choose from a limited set of idealized arc forms in developing a treatment plan for a patient, which may not consider, for example, contact surfaces between the patient's upper and lower teeth.

The disclosures herein may result in improved aesthetics, improved functionality, and/or better patient experiences. The disclosures herein can enable a practitioner to better consider the overall architecture of a patient's teeth (and their placement relative to one another, for example), which can result in improved outcomes.

In some embodiments, the systems, methods, and devices described herein are configured to identify one or more parameters that can be used to evaluate, recreate, and/or alter the positioning of structures that are poorly positioned or missing. While in some embodiments, the lost or poorly positioned structures are teeth, the disclosure herein is not limited to teeth. For example, the disclosures herein can be applied to other structures such as, for example, roots and/or bone structures. For example, the basal bone of the maxilla or mandible that supports the teeth may not fit with the ideal determined positions of the patient's teeth. Accordingly, in some embodiments, the processes herein can be used to determine a new position of the bone, which may be used by a maxillofacial surgeon to surgically reposition the bone.

Teeth can be organized in a system and can have non-random positions and/or non-random shapes. It can thus be important to consider morphometric parameters that can be specific to a patient when adding, recreating, moving, and or realigning teeth. Morphometric parameters can include, for example, lip position, arch location, bone location, and so forth. In some embodiments, parameters such as static occlusion and/or dynamic occlusion may be considered in determining the placement of teeth or a protheses. Determining these parameters can be a difficult and/or time-consuming process for a practitioner, especially in the case of major rehabilitations or complex diagnoses. For example, treating an edentulous patient can be especially challenging as the patient has no existing teeth. Aspects of the present disclosure may be used to make it easier to evaluate, recreate, and/or straighten lost of poorly positioned structures.

In some embodiments, a tooth library (e.g., a collection of predefined tooth shapes) can be created that aids in the automatic positioning of teeth. For example, a library can aid in automatic positioning of teeth using morphometric data of the patient. In some embodiments, a library can include representations of a patient's teeth, representations of artificial teeth, or both. For example, the patient's actual teeth can be used when planning an orthodontic treatment, while artificial teeth (or artificial teeth and the patient's own teeth) can be used when planning a prosthetic treatment. In some embodiments, both the patient's own teeth and artificial teeth can be used when planning an orthodontic and/or prosthetic treatment, for example to help ensure that the prosthetic teeth fit well with the patient's existing teeth. In some embodiments, a practitioner can modify the positioning of one or more teeth. In some embodiments, an artificial intelligence or machine learning (AI/ML) model can be used to improve the positioning of one or more of the patient's teeth, artificial teeth, or both. In some embodiments, positioning may be adjusted based at least in part on the patient's preferences, the country or region in which the patient resides or where the orthodontic or prosthetic treatment is performed, and so forth.

In some embodiments, positioning of one or more teeth can be automatic or partially automatic. In some embodiments, the positions of one or more teeth can be modified to change aesthetics. In some embodiments, the positions of one or more teeth can be modified to change dynamics, for example to improve functionality such as eating or speaking. In some embodiments, the orientation of one or more posterior teeth can be modified, for example with respect to a frontal plane, a sagittal plane, or both. In some embodiments, the inclination of one or more teeth can be modified. Characteristic points of each tooth can be connected to the corresponding arcs that are used to define the helix. In some cases, steeper inclinations can increase contact during movement. In some embodiments, automatic, partially automatic, or manual movements can be made with respect to, for example, canine guidance, progressive function, group function, generally balanced occlusion, or any combination of these. In some embodiments, the teeth can be fitted on a double helix. Thus, adjusting the shape of the double helix can change a guidance function of the teeth. A double helix can be constructed from multiple portions or faces. As one example, a fitting arc can be connected with an aesthetic arc for premolars and molars, thereby creating a face. The face can have an inclination with respect to the axio-orbital plane. In some embodiments, such a face can be parallelized with the axio-orbital plane such that the maxillary and mandibular molars and premolars to not make contact during excursive movement (e.g., laterotrusion left and/or right), but contact can be maintained on the canines.

In some embodiments, if jaw motion data is available, contact relations between the teeth can be determined, and contact points can be determined after positioning the teeth on the double helix. In some embodiments, the contacts may be undesirable, and the design of the double helix and tooth positioning thereon can be modified to alter the position and/or the shape of teeth, thereby modifying the contacts between the mandibular and maxillary teeth. In some embodiments, an AI/ML model can be trained to output desirable positioning of the teeth, shapes of the teeth, and so forth. Additional details with regard to contact relations and contact points between teeth are provided later within this disclosure.

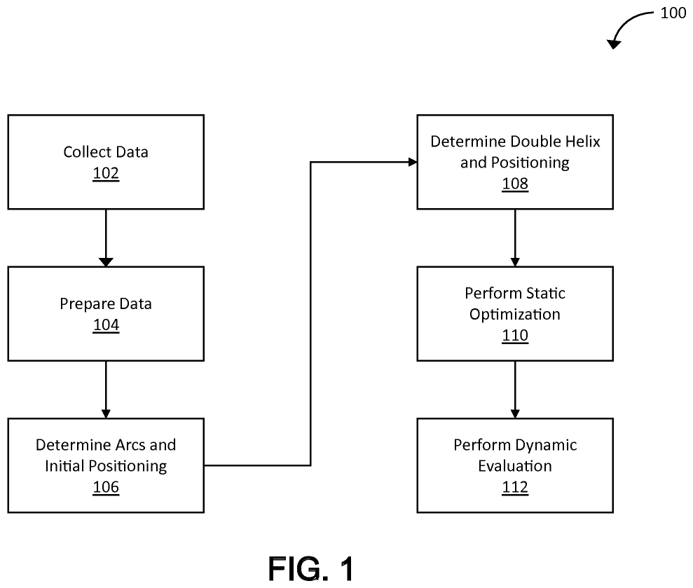

FIG. 1 shows an example process 100 for generating a tooth positioning plan for a patient according to some embodiments. The steps shown in FIG. 1 are merely examples. In some embodiments, a process can include more steps, fewer steps, and/or the steps can be performed in an order different from that shown in FIG. 1.

At block 102, a practitioner can collect data about the patient, such as facial and tooth information. At block 104, a system can be used to prepare the data for creating a treatment plan or designing a smile. At block 106, the system can determine one or more arcs and can position the teeth on the one or more arcs. At block 108, the system can be used to generate a geometric structure such as a double helix. At block 110, the system can perform static optimization of the patient's teeth. At block 112, the system can perform dynamic evaluation of the patient's teeth, jaw, and so forth, which may result in further refine of the positioning of the teeth. Each of these steps is discussed in more detail below.

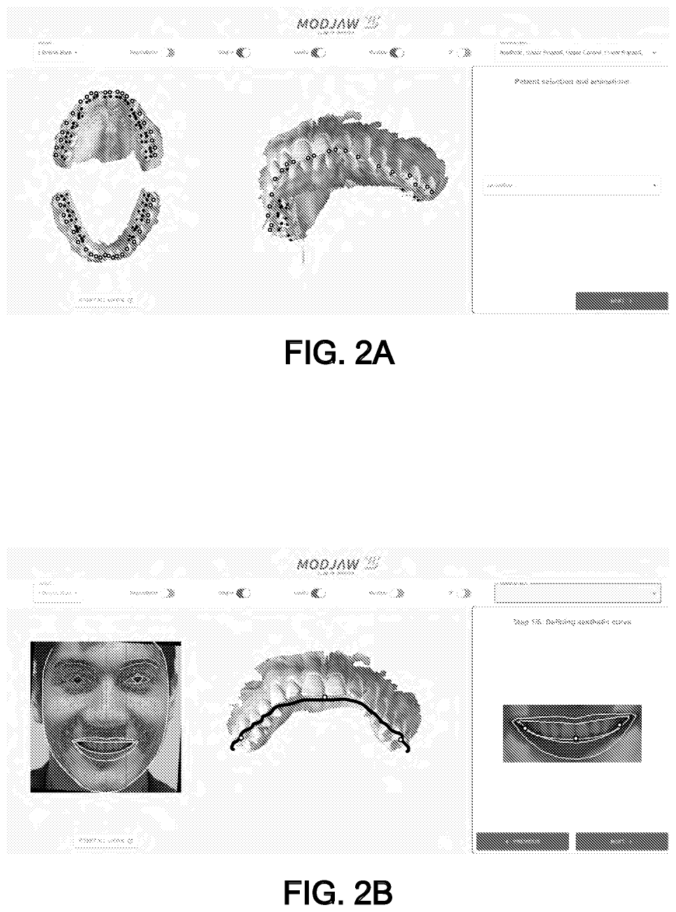

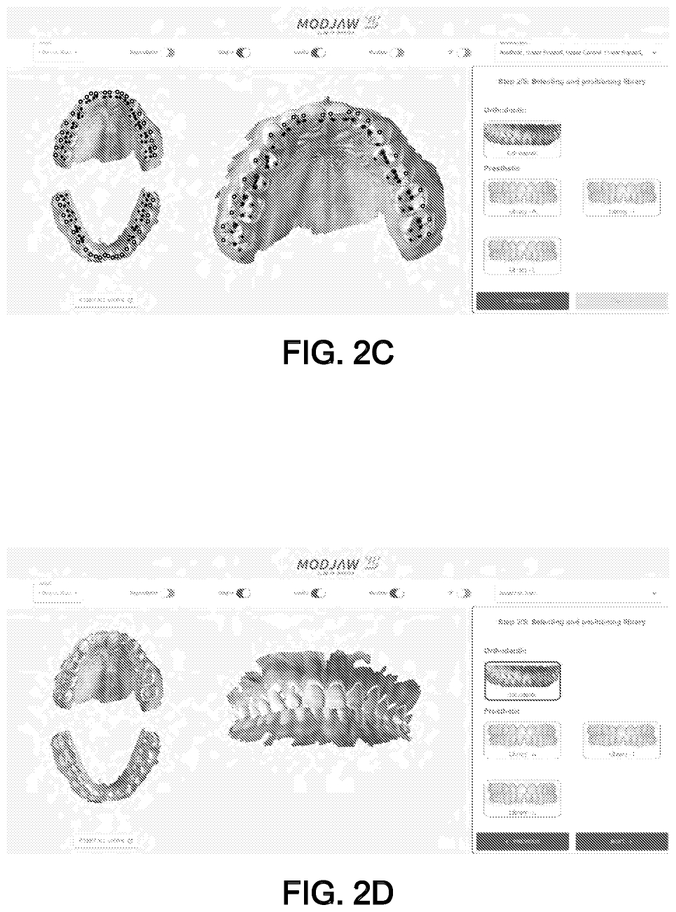

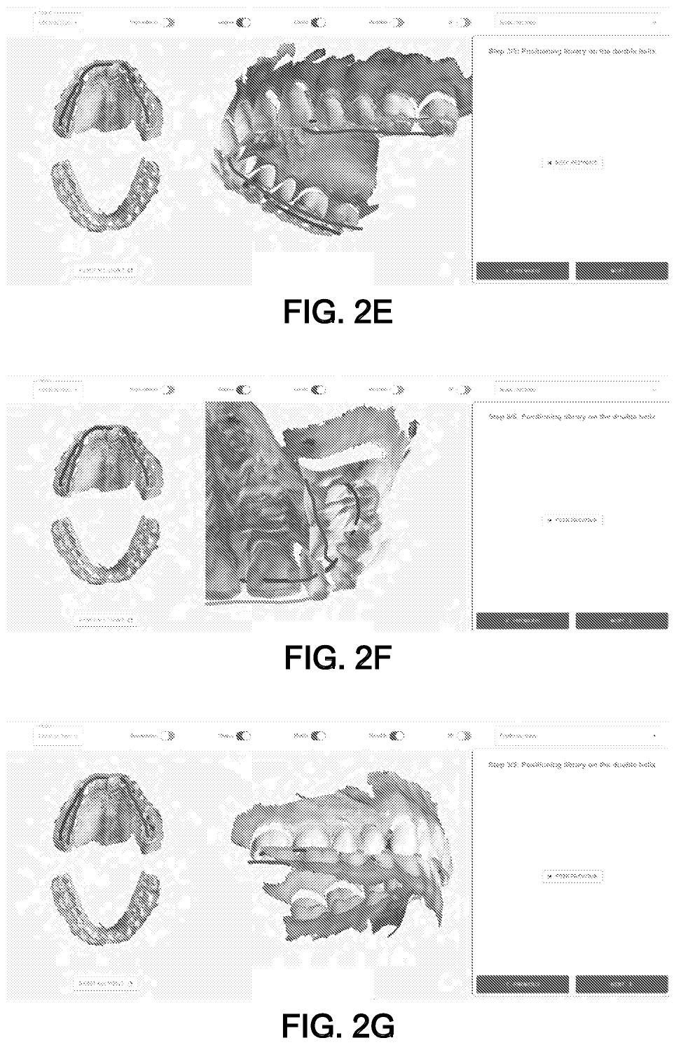

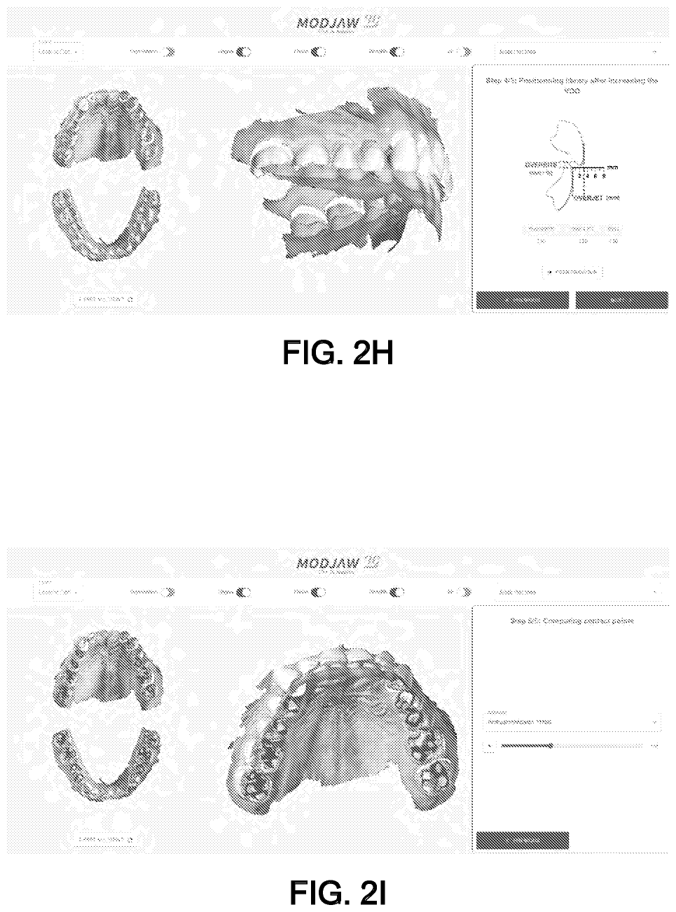

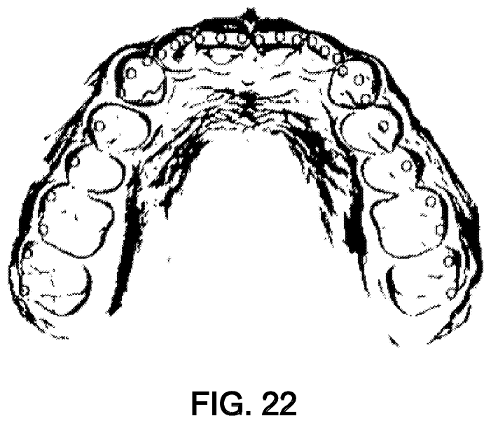

FIGS. 2A-21 illustrate an example implementation of a process for orthodontic treatment planning according to some embodiments. In FIG. 2A, a user can select a patient whose treatment is to be planned, and the multiple arcs can be associated with the patient, for example an aesthetic arc (open circles), centering arc (solid circles), and fitting arc (crosses). For greater clarity, example points and arcs that correspond to points and arcs shown in FIGS. 2A-21 and 3A-3K are shown in greater detail in FIGS. 22-28. In FIG. 2B, the user can define an aesthetic curve or arc. In FIGS. 2C and 2D, the user can select and position a library. As illustrated in FIGS. 2C and 2D, the user can select from an orthodontic library (e.g., the patient's own teeth) or from one or more prosthetic libraries. In FIGS. 2E-2G, the user can position the library on the double helix. In FIG. 2H, the user can position the library after modifying the vertical dimension of occlusion to achieve desired overbite and/or overjet values. In FIG. 2I, the user can compute contact relations between maxillary and mandibular teeth, as indicated by the shade areas of the teeth. In FIG. 2K, the user can compute contact points between the upper and lower teeth during jaw motion and/or at particular jaw positions.

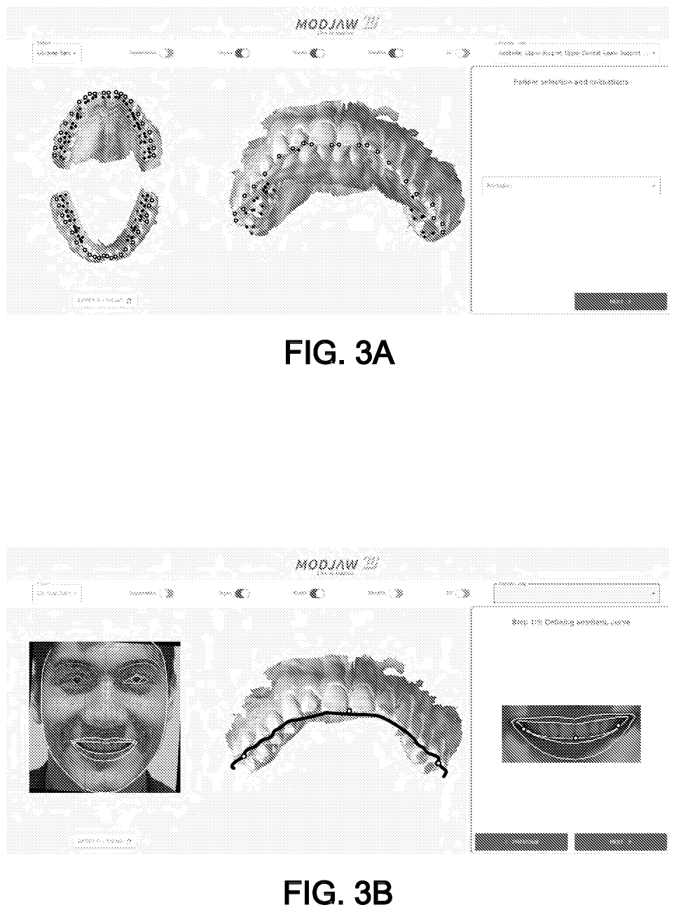

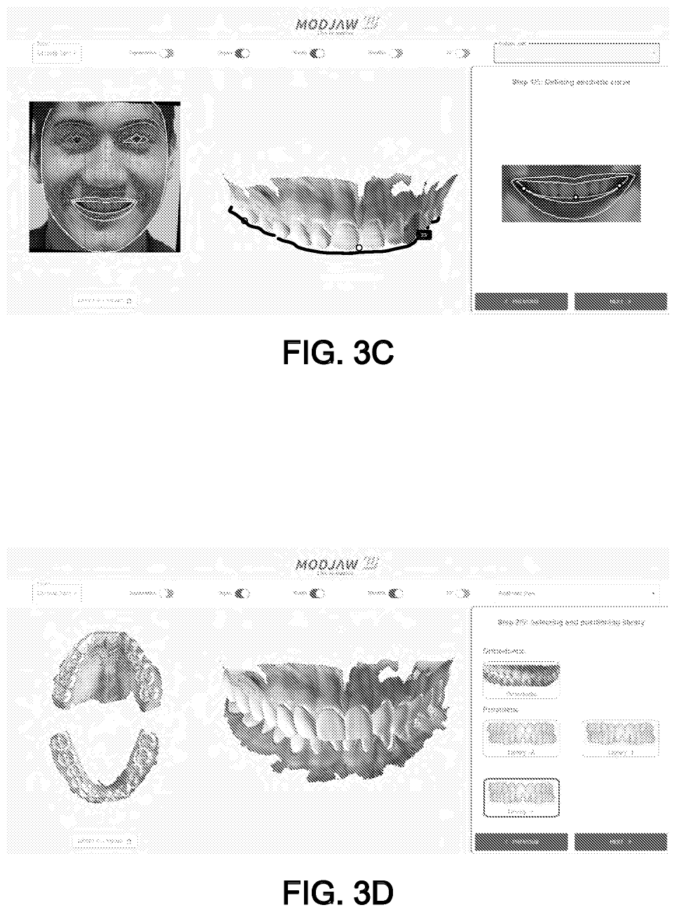

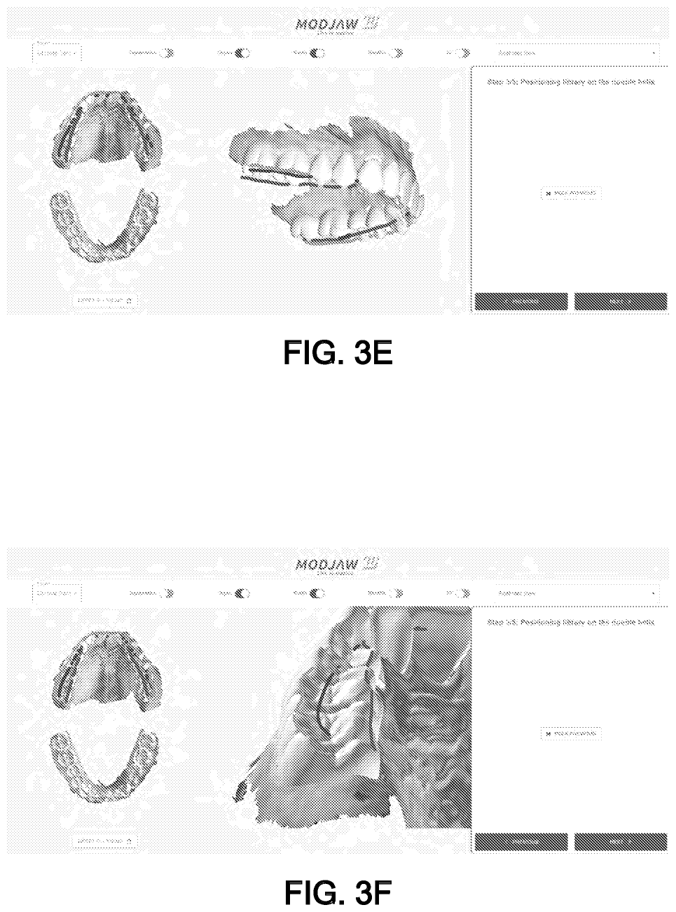

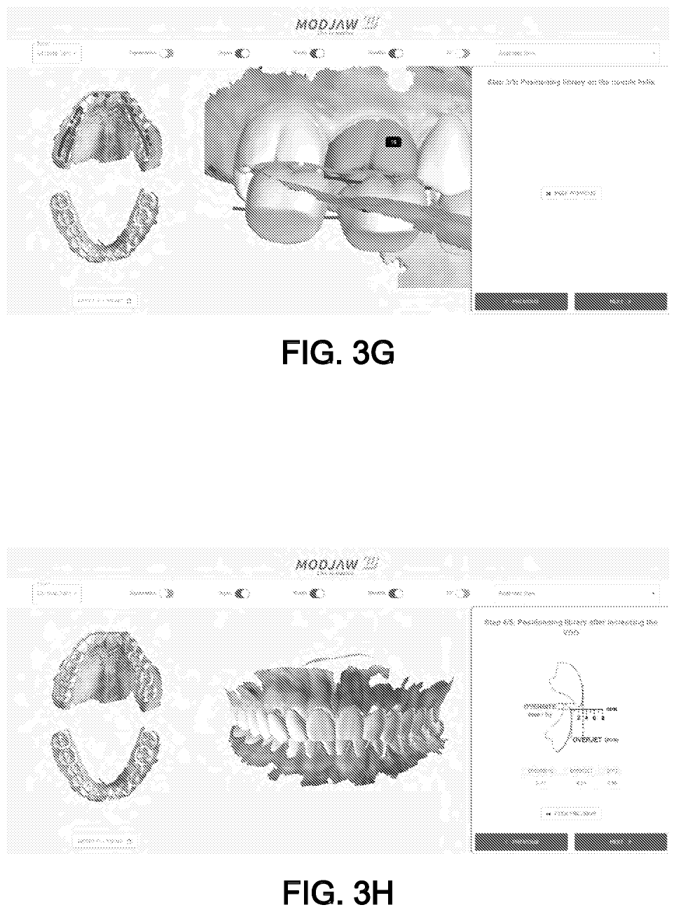

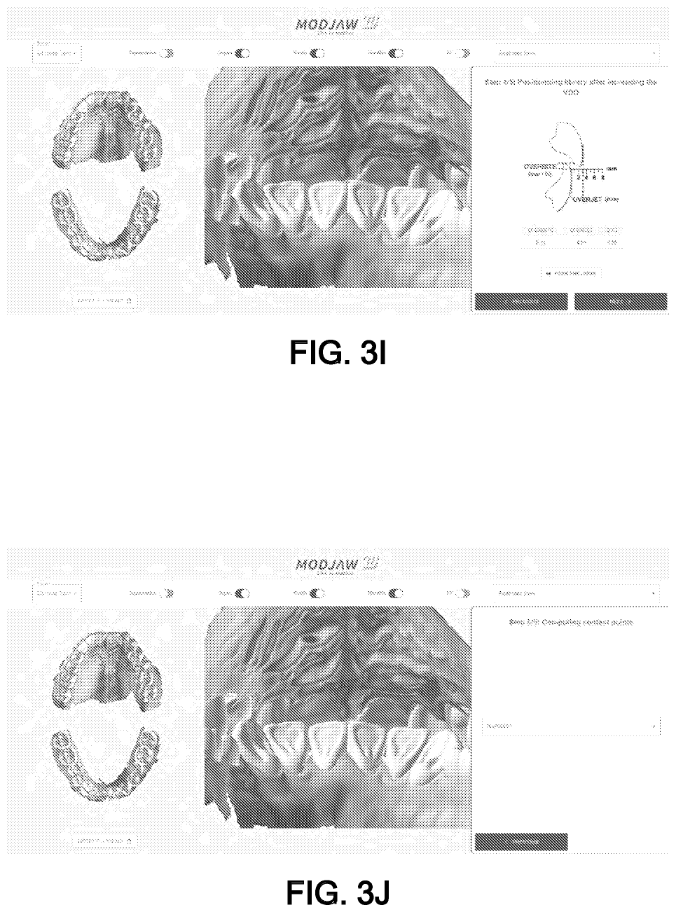

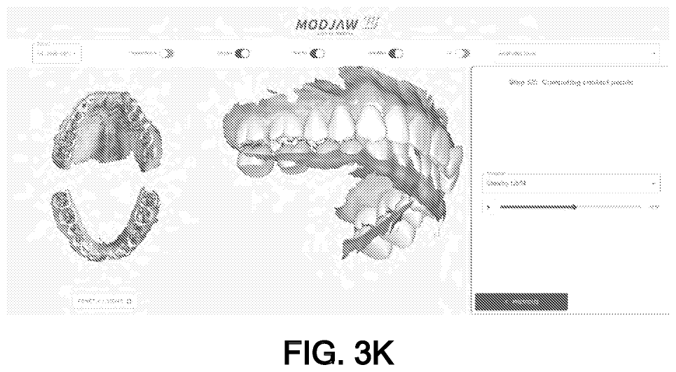

FIGS. 3A-3K illustrate an example implementation of a process for prosthetic treatment planning according to some embodiments. The process shown in FIGS. 3A-3J can be broadly similar to the orthodontic process depicted in FIGS. 2A-21. In FIG. 3A, a user can select a patient for treatment planning. In FIGS. 3B and 3C, the user can define an aesthetic curve for the patient. In FIG. 3D, the user can select a library. In FIGS. 3E-3G, the user can position the library on the double helix. In FIGS. 3H and 3J, the user can adjust the relative positioning of the teeth to achieve desired overbite and/or overjet. In FIGS. 3J and 3K, the user can use the system to compute and visualize contact points between the maxillary and mandibular teeth during jaw motion and/or at particular jaw positions, for example as indicated by the shaded areas of the teeth in FIG. 3K.

### Data Collection and Preparation

As mentioned briefly above, better treatment outcomes can be achieved if a practitioner considers a fuller set of data about a patient. Preferably, a practitioner considers both aesthetic and functional aspects when determining a treatment plan. Thus, it can be advantageous to collect a considerable amount of data about the patient's teeth, facial structure, jaw alignment, temporo-mandibular joint motion, jaw movement, bone structure, and so forth.

In some embodiments, the patient's facial and/or jaw movements can be considered when formulating a diagnosis and/or a treatment plan. In some cases, motion capture systems can be used to map the movement of the patient's face and jaw during actions such as speaking, smiling, and chewing. For example, markers may be applied to the patient's face and the movements tracked using an infrared camera. In some embodiments, specialized hardware and/or software can be used for recording and/or simulating a patient's jaw movements, for example as described in U.S. Pat. No. 10,265,149, issued Apr. 23, 1919, the contents of which are hereby incorporated by reference in their entirety herein.

However, some providers may not have access to specialized equipment for facial motion capture. Accordingly, in some embodiments, providers can capture facial motion without the need for specialized equipment, for example using consumer imaging hardware.

The captures of the patient's face and movements can be used in combination with a 3D representation of the patient's teeth, bones, and/or other anatomical features as part of a process to determine optimal placement of the teeth. The 3D representation of the patient's teeth may be obtained from, for example, an intraoral scanner, a lab scan of a mold of the patient's teeth, a cone-beam computed tomography scan, and so forth. These technologies are commonly available to dental practitioners. Preferably, the teeth may be segmented as described more fully below. For example, each tooth and the gums may be treated separately, teeth may be divided into groups and the groups treated separately, or individual teeth may be divided into more than one segment. In some cases, different segmentations (e.g., partial tooth, full tooth, multiple teeth) can be used for developing a treatment plan for a single patient. In some cases, the information for each tooth as well as the gingiva may be stored in separate files, though this need not be the case.

In some embodiments, certain points and parameters such as, for example, the condyles, the location of the arches in relation to the lips, the size of the arches in relation to the dimensions of the mouth, and so forth may be manually, partially manually, or automatically set. Preferably, the alignment of the patient's face and teeth may be performed automatically.

In some embodiments, data may be collected that describes a patient's 3D dental architecture. For example, dental architecture data can include information about various angles and/or reference planes (e.g., condylar inclination angle, axio-orbital plane, etc.), mandibular movement, lip position, and so forth. In some embodiments, the data can include information that describes arch position, for example in relation to temporo-mandibular joints. In some embodiments, a practitioner may use this data to develop a treatment plan that is tailored to an individual patient. In some embodiments, the data can include descriptions of teeth position, static positioning of the jaw, and/or dynamic movements. In some embodiments, the data may describe aesthetic aspects, functional aspects, or both.

As briefly mentioned above, in some embodiments, individual teeth may be segmented and/or individually identified. For example, a system can be configured to automatically, semi-automatically, or manually (e.g., relying on user input) segment the teeth. As discussed in more detail below, various methods can be used for segmenting the teeth. In some embodiments segments may be individual teeth, although this is not necessarily the case. For example, a segment can include part of a tooth, multiple teeth, parts of multiple teeth, a combination of whole teeth and partial teeth, etc. In some embodiments, when a segment comprises an individual tooth, the system can automatically designate the tooth with its name or identifier, e.g., according to the ISO 3950 standard, the Universal Numbering System, Palmer notation, and so forth. For example, a machine learning algorithm can be trained to automatically identify teeth and assign an appropriate designation (e.g., “canine 13” for the patient's upper right canine, according to the ISO 3950 standard).

In some embodiments, metadata can be determined that describes, for example particular points, areas, features, and so forth of the dental surface. In some embodiments, a virtual surface (e.g., a double helix) can be created, and the geometry of the virtual surface can be used to indicate and/or determine the positioning of the teeth and/or a zone of confrontation between one or more teeth of the mandibular arch and the maxillary arch. For example, the zone of confrontation can describe the contact points, angles, and so forth between mandibular and maxillary teeth. The zone of confrontation can be defined as the zone where the mesh of one tooth comes into contact with the mesh of another tooth. In some embodiments, the contact may be between the teeth of the mandibular and maxillary arches. The zone of confrontation can include static (e.g., jaw closed and stationary) occlusion, dynamic occlusion (e.g., contacts made when the jaw is moving), or both.

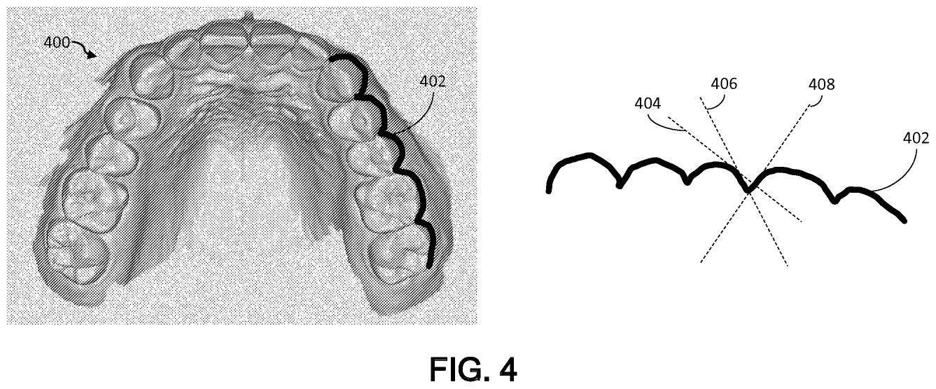

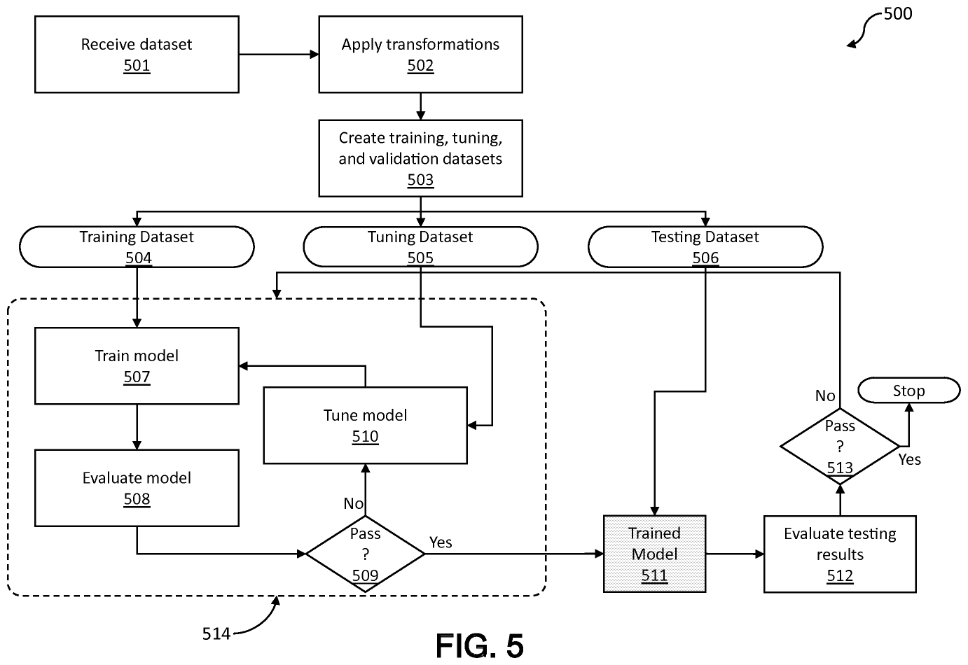

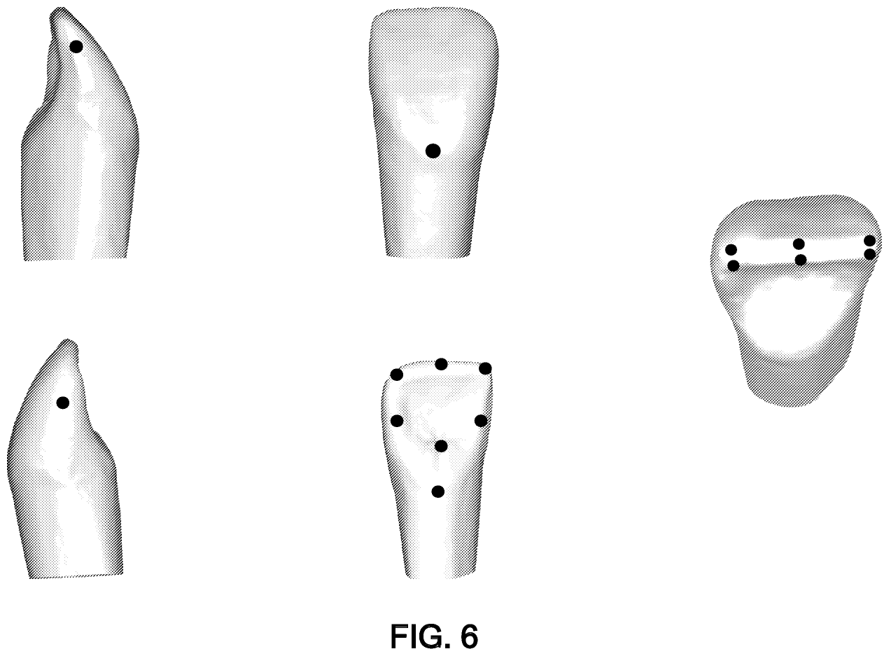

In some embodiments, the systems, methods, and devices described herein can provide automated and/or semi-automated solutions for determining the boundaries between teeth, the gumline, and so forth. In some embodiments, three-dimensional dental arches may be represented by point clouds, meshes, and so forth. In some embodiments, a representation of a dental arch can be segmented or sub-divided into subunits that can be given specific names or identifiers. For example, a subunit can be a single tooth, more than one tooth, part of a tooth, and so forth. In some embodiments, segmentation can be based on color discrimination, geometric transition variations, and so forth. For example, a gumline can be identified by a change in color or teeth can be distinguished from one another by looking for sharp changes in the slope of a profile or changes in the sign of a slope. For example, FIG. 5 illustrates a profile view in which the magnitude of the slope sharply increases as the interface between two teeth is reached, and the sign of the slope rapidly changes at or near the interface between two teeth. As shown in FIG. 4, teeth 400 can have a profile 402. The slope 404, which is relatively far from the interface between teeth, can be shallower than the slope 406, near the interface between two teeth. The slope 408 can be of a different sign than the slope 406 and can correspond to another tooth. For example, as shown in FIG. 4, the slope 406 can be relatively large and negative, while the slope 408 can be relatively large and positive. In some embodiments, the interface between teeth can be determined as an inflection point where the slope changes from negative to positive. In some embodiments, artificial intelligence (AI) and/or machine learning (ML) models can be used for segmentation. In some embodiments, the boundaries or edges of teeth can be determined based on the local 3D curvature. For example, a boundary may be indicated by a high variation in slopes in a relatively small area. In some embodiments, AI and/or ML models can improve efficiency, accuracy, or both.

FIG. 5 depicts a flow chart for training an artificial intelligence or machine learning model according to some embodiments. The training process depicted in FIG. 5 can be used for training models to be used in a variety of applications. For example, the training process 500 can be used to train a model to identify arcs (e.g., aesthetic arcs, centering arcs, fitting arcs, etc., as described herein), segment teeth, position teeth, and so forth. In some embodiments, a model can be trained to identify prosthetic libraries that may be used for treating a patient, for example to choose the most appropriate library or libraries from a set of standard libraries. In some embodiments, a model can be trained to generate a tooth library. At block 501, the system may receive a dataset that includes various information for use in training a model, such as facial captures, jaw motion captures, tooth positioning data, images of teeth and/or gums, and so forth. At block 502, one or more transformations may be performed on the data. For example, data may require transformations to conform to expected input formats, for example to conform with expected date formatting, to conform to a particular tooth numbering system (e.g., Universal Numbering System, FDI World Dental Federation notation, or Palmer notation). In some embodiments, the data may undergo conversions to prepare it for use in training an AI or ML algorithm, which typically operates using data that has undergone some form of normalization or other alteration. For example, categorical data may be encoded in a particular manner. Nominal data may be encoded using one-hot encoding, binary encoding, feature hashing, or other suitable encoding methods. Ordinal data may be encoded using ordinal encoding, polynomial encoding, Helmert encoding, and so forth. Numerical data may be normalized, for example by scaling data to a maximum of 1 and a minimum of 0 or −1. Image data can undergo various transformations. For example, a channel value may be converted from a 0-255 range to a 0-1 range, image resolution can be set to standardized values, etc.

At block 503, the system may create, from the received dataset, training, tuning, and testing/validation datasets. The training dataset 504 may be used during training to determine variables for forming a predictive model. The tuning dataset 505 may be used to select final models and to prevent or correct overfitting that may occur during training with the training dataset 504, as the trained model should be generally applicable to a broad spectrum of patients, rather than to the particularities of the training data set (for example, if the training data set is biased towards patients with relatively high or low bone density, wide or narrow dental arches, etc.). The testing dataset 506 may be used after training and tuning to evaluate the model. For example, the testing dataset 506 may be used to check if the model is overfitted to the training dataset. The system, in training loop 514, may train the model at 507 using the training dataset 504. Training may be conducted in a supervised, unsupervised, or partially supervised manner. At block 508, the system may evaluate the model according to one or more evaluation criteria. For example, the evaluation can include determining whether segmentation is accurate, determining whether suggested libraries are suitable, determining whether suggested arches are identified appropriately, determining whether are teeth are suitably positioned, or any other criteria as may be desirable At block 509, the system may determine if the model meets the one or more evaluation criteria. If the model fails evaluation, the system may, at block 510, tune the model using the tuning dataset 505, repeating the training 507 and evaluation 508 until the model passes the evaluation at block 509. Once the model passes the evaluation at 509, the system may exit the model training loop 514. The testing dataset 506 may be run through the trained model 511 and, at block 512, the system may evaluate the results. If the evaluation fails, at block 513, the system may reenter training loop 514 for additional training and tuning. If the model passes, the system may stop the training process, resulting in a trained model 511. In some embodiments, the training process may be modified. For example, the system may not use a testing dataset 506 in some embodiments. In some embodiments, the system may use a single dataset. In some embodiments, the system may use two datasets. In some embodiments, the system may use more than three datasets. In some embodiments, the model may not use a tuning dataset. For example, the model may have a training dataset and a testing dataset.

As discussed briefly above, metadata can be used to describe properties of individual teeth, segments, and so forth. In some embodiments, metadata can describe dental morphology. In some embodiments, metadata can include information related to structures such as, for example, cusps, fossae, ridges, grooves, zones of inflection, zones of greater contour, and so forth. In some embodiments, the metadata can be manipulated to move a segment in space, to deform a segment, to resize a segment in whole or in part, and so forth. In some embodiments, metadata for different areas can be considered separately or together.

In some embodiments, metadata may be determined for a patient's existing teeth. In some embodiments, metadata can be determined for, as an example, a library of standardized or artificial teeth, for example if a treatment plan includes replacing a diseased or missing tooth with an artificial tooth. In some embodiments, AI/ML models can be used to determine metadata for existing teeth. In some embodiments, AI/ML models can be used to recognize, process, etc., metadata for existing teeth. In some embodiments, an AI/ML model can be trained using a database of teeth that has been manually annotated by humans. For example, ridges, cusps, pits, dimples, furrows, zones of inflection, zones of greater contour, and so forth can be manually annotated in a training data set such that an AI/ML model can be trained to recognize one or more of these features. An AI model can be updated periodically, for example by providing additional annotated data.

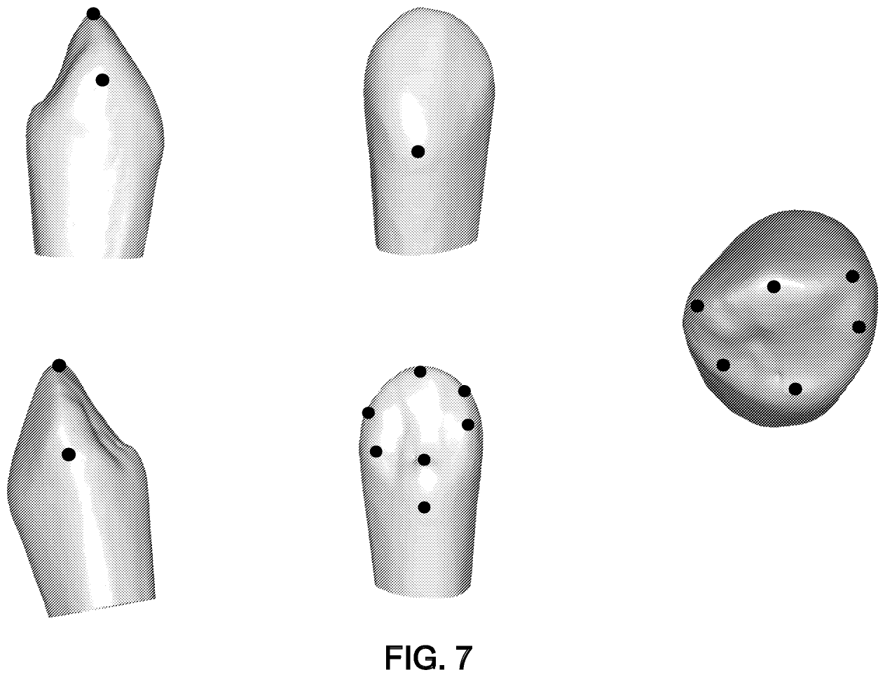

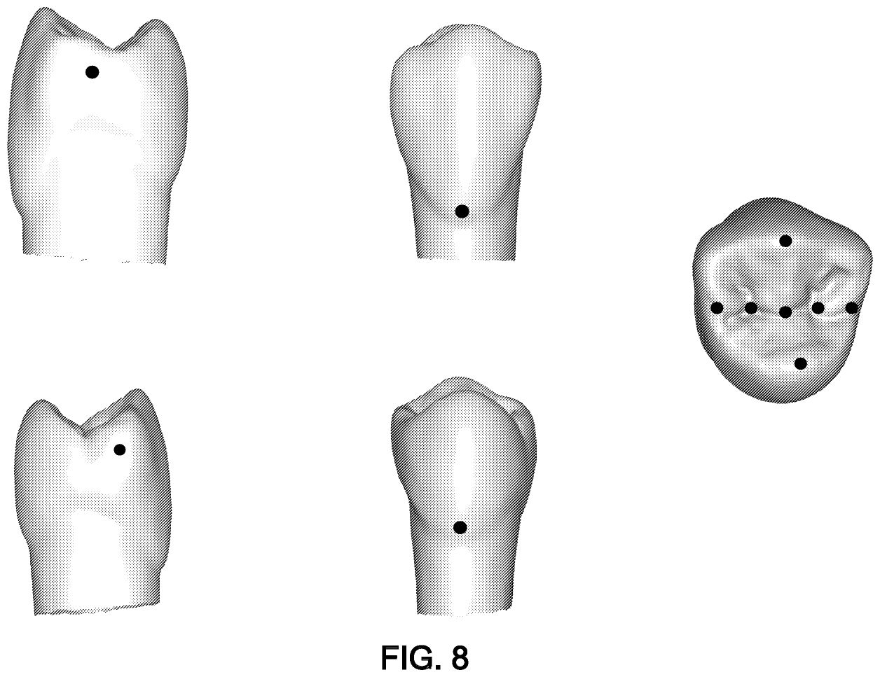

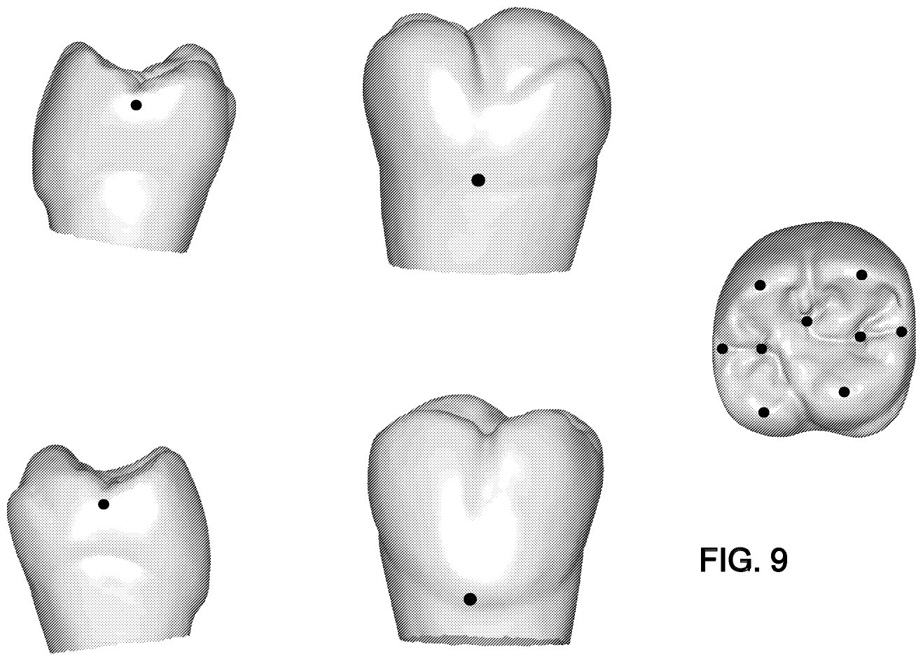

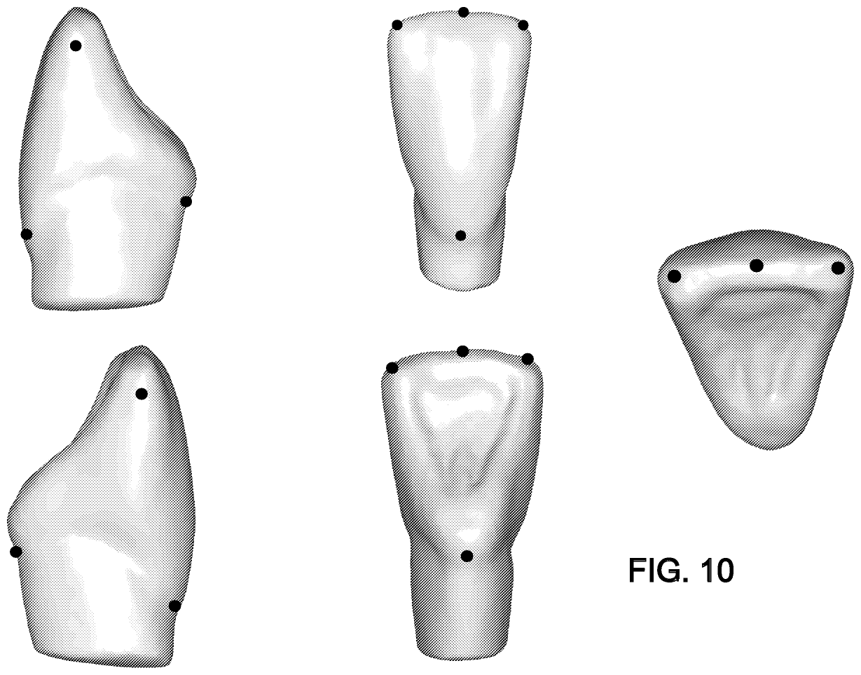

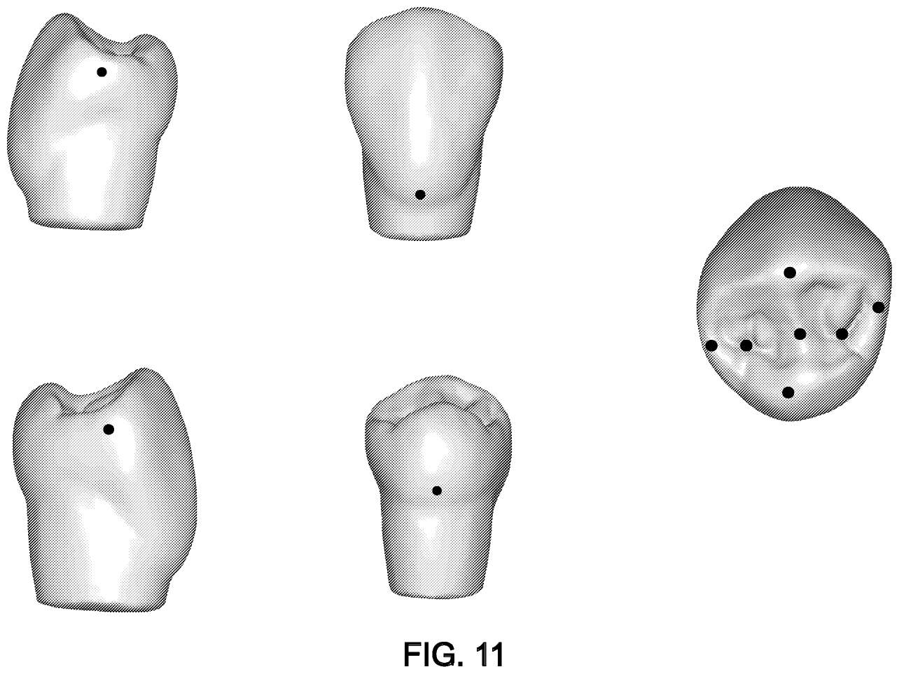

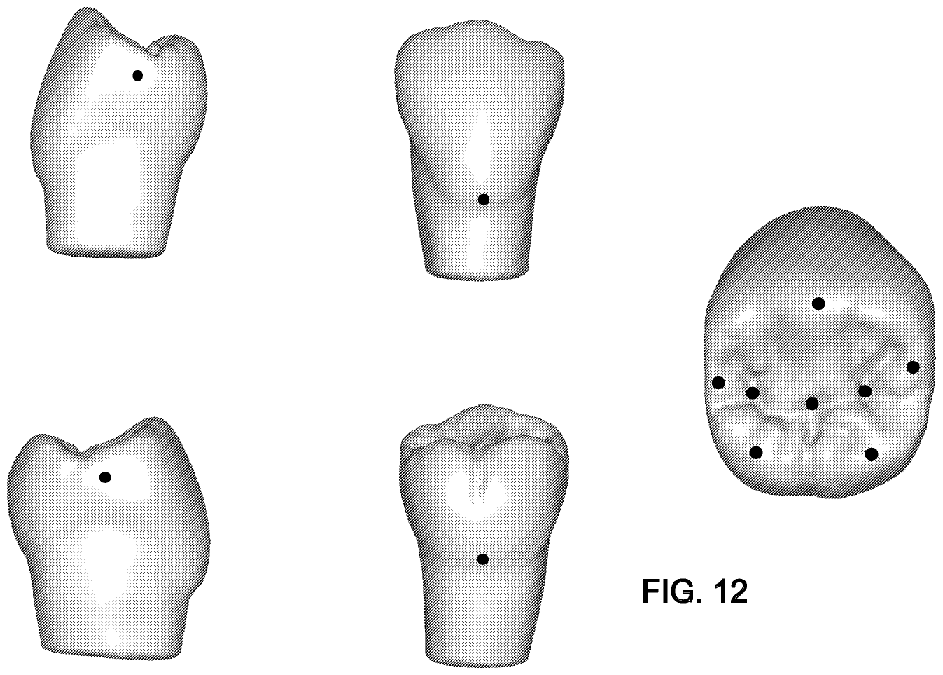

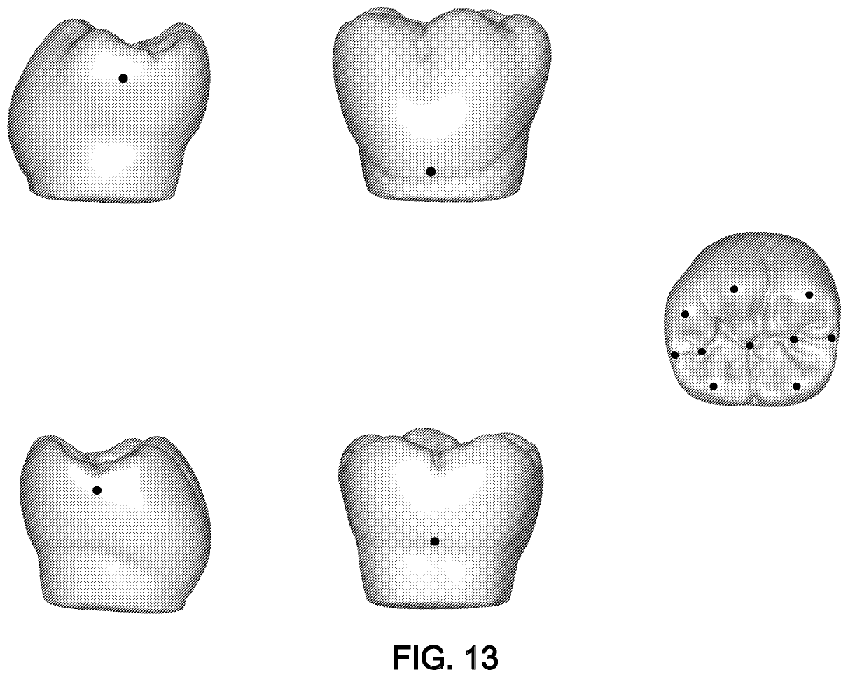

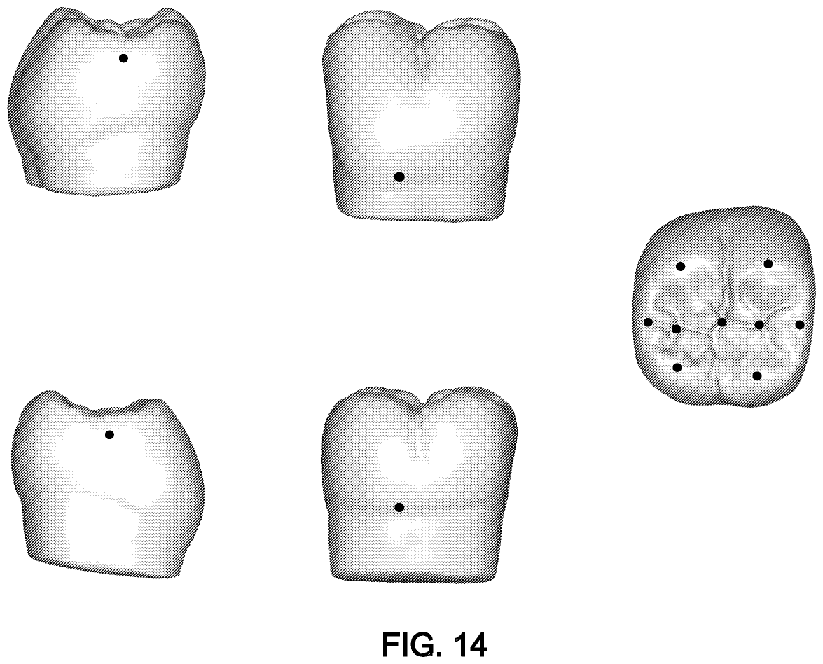

FIGS. 6-14 illustrate examples of annotations of teeth according to some embodiments. FIG. 6 illustrates annotation of the maxillary incisors 11, 12, 21, and 22 according to some embodiments. FIG. 7 illustrates annotation of the maxillary canines 13 and 23 according to some embodiments. FIG. 8 illustrates annotation of the maxillary premolars 14, 15, 24, and 25 according to some embodiments. FIG. 9 illustrates annotation of the maxillary molars 16, 17, 26, and 27 according to some embodiments. FIG. 10 illustrates annotation of the mandibular incisors 31, 32, 41, and 42 according to some embodiments. FIG. 11 illustrates annotation of the mandibular premolars 34 and 44 according to some embodiments. FIG. 12 illustrates annotation of the mandibular premolars 35 and 45 according to some embodiments. FIG. 13 illustrates annotation of the mandibular molars 36 and 46 according to some embodiments. FIG. 14 illustrates annotation of the mandibular molars 37 and 47 according to some embodiments. In some embodiments, annotations can include, for example and without limitation, mesio-vestibular cusps, mesio-palatine cusps, disto-vestibular cusps, disto-palatine cusps, mesio-lingual cusps, disto-lingual cusps, vestibular cusps, palatal cusps, distal cusps, mesial incisal edges, central incisal edges, distal incisal edges, canine tips, mesial crests, distal crests, mesial dimples, central pits, distal dimples, cingula, main grooves, mesial contact points, distal contact points, mesial points, distal points, middle cervical buccal points, middle cervical lingual points, middle cervical palatal points, and so forth.

### Arc Determination and Positioning

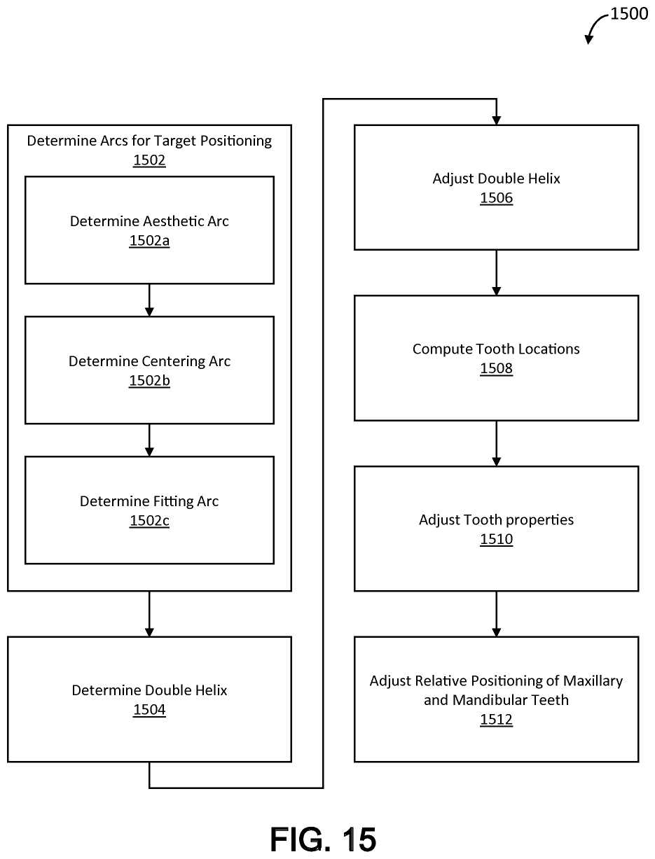

FIG. 15 illustrates an example process 1500 for determining a treatment plan for a patient. Additional details for each step of the process 1500 are described herein. At block 1502, a system can be configured to determine arcs for target positioning of the patient's teeth. At 1502a, the system can be configured to determine an aesthetic arc. As described in more detail below, the aesthetic arc can be built using specific points on the teeth (e.g., ridges, cusps, pits, edges, etc.). In some embodiments, a practitioner can provide inputs that define the expected final position (e.g., set using control points adjusted on a 2D picture of the patient and/or on a 3D model of the patient). In some embodiments, teeth edges can be used for building the aesthetic arc and can be automatically detected from a picture, face scan, or other patient data. In some embodiments, facial landmarks can be used to determine the aesthetic arc. In some embodiments, 2D images may be used and the system can map from two dimensions to three dimensions. In some embodiments, filtering and/or smoothing algorithms can be used to smooth the aesthetic arc (or other arcs as described herein).

At 1502b, the system can be configured to determine a centering arc, which can be based at least in part on the aesthetic arc determined at block 1502a. At block 1502c, the system can be configured to determine a fitting arc. In some embodiments more arcs, fewer arcs, or different arcs can be determined. The various arcs that can be used for determining positioning of teeth and other properties are described in more detail below. At block 1504, the system can determine a double helix based on the arcs determined at block 1502. At block 1506, the system can be configured to adjust the double helix. For example, the system may provide automated, semi-automated, and/or manual adjustment functionality (e.g., a practitioner may, in some embodiments, manually edit the double helix or one or more arcs used to compute the double helix). At block 1508, the system can be configured to compute tooth locations based on the double helix and/or the aesthetic arc. At block 1510, the system can be used to automatically, semi-automatically, and/or manually adjust the location, orientation, shape, and/or size of one or more teeth using the double helix. At block 1512, the system can be configured to adjust the relative locations of mandibular and maxillary teeth, for example by taking into account contact relations between the teeth, dynamic behavior of the teeth and/or jaw, and desired overbite and/or overjet characteristics.

In some embodiments, an aesthetic arc can be a 3D line that joins the buccal edges of the maxillary teeth, incisal edges, canine tips, buccal cusps, and the like. In some embodiments, an aesthetic arc can be based on a freehand line drawn by a practitioner, a line drawn with the aid of a pre-existing dental preform, a line generated by a computer system, and so forth. In some embodiments, a previously-taken photo can be superimposed on a 3D model and can help to position the aesthetic line. In some embodiments, a stopping point of the aesthetic line can correspond to a location of the posterior edge of the last teeth of the arc, for example a second molar. In some embodiments, a double helix geometric shape can be used, and the aesthetic arc can define the external limit of the double helix. In some embodiments, a double helix can have a first torsion that describes the inclination of the dental surfaces of the teeth and a second torsion that corresponds to the shape of the dental arch.

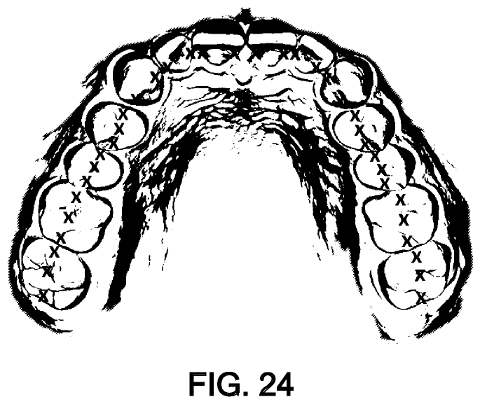

Multiple arcs can be associated with a patient's teeth, as will be explained in more detail below, for example in FIG. 24. In some embodiments, an aesthetic arc can be used to define an external limit of the double helix. Additional arcs, such as a fitting arc and centering arc can be used to further define the double helix. In some embodiments, determination of the external limit of the double helix via the aesthetic arc can be an early or initial step in determining the double helix. As described in more detail below, in some embodiments, at least in part based on the position of the aesthetic arc, other arcs can be determined.

In some embodiments, an aesthetic arc for a patient can be calculated from a maxilla mesh and a patient face image, for example as captured by an intraoral scanner and a facial scanning device (which can be a specialized device or a non-specialized device such as a smartphone, tablet, depth-sensing camera, and so forth). In some embodiments, landmarks between the maxilla mesh and the facial image can be mapped. The intrinsic parameters of the camera used to capture the facial image can also be considered. For example, it may be important to know the focal length of the camera. In some cases, it may be useful to know the resolution of the camera or other parameters of the camera. In some embodiments, information about the camera (such as the focal length) can be used to remove distortions such as a fisheye effect that can result from capturing images with a wide angle lens. In some embodiments, a practitioner can define control points for use in calculating the aesthetic arc. In some embodiments, three control points can be used, although the number of control points is not necessarily limited. In some embodiments, control points can have initial positions. In some embodiments, control points can have both initial positions and modified positions.

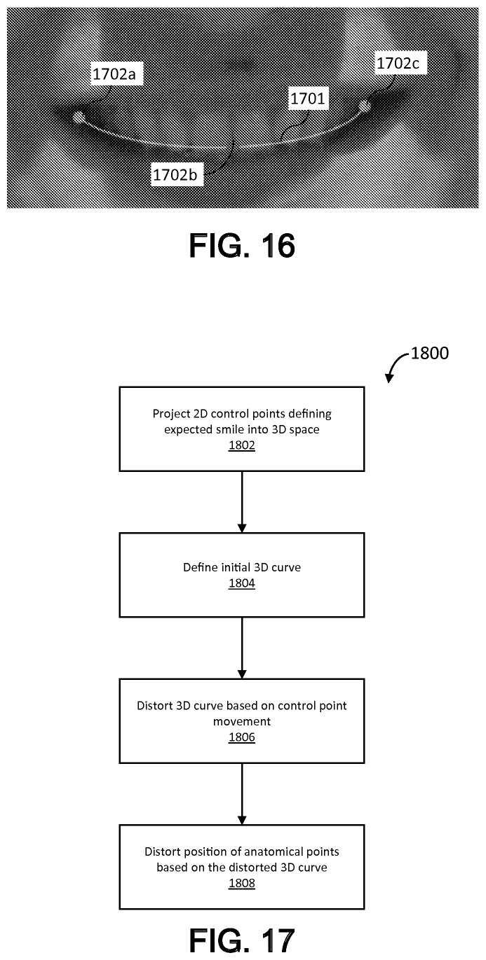

As depicted in FIG. 16, in some embodiments, an aesthetic arc 1601 may comprise three control points 1702a-c. A user interface of a system may show the aesthetic arc 1601 and the control points 1702a-c overlaid on an image of the patient's face and/or teeth. In some embodiments, a practitioner can manipulate the control points to define an expected and/or desired position on the position on the patient's smile. In some embodiments, the control points may correspond to, for example, molars 16 and 26 and a midpoint between the incisors 11 and 21, although other control point placements are possible.

In some embodiments, the initial design of the aesthetic arc as depicted in FIG. 16 can be performed using a 2D projection of the patient's teeth. In some embodiments, a system can be configured to detect landmarks on a picture of the patient's face. For example, the system can use a picture of the patient and can be configured to detect the patient's face, identify one or more landmarks, and draw a smile line and/or other reference points and/or lines that are useful for designing the patient's smile.

While design can be performed using 2D projections, preferably a dental diagnostic and/or treatment plan should consider the 3D positioning of the patient's teeth. FIG. 17 depicts an example process 1700 for determining a 3D curve according to some embodiments. The process shown in FIG. 17 can, in other embodiments, include fewer or additional steps. In some embodiments, it can be advantageous to map between a 2D image (e.g., a photo of the patient's face) and a 3D capture of the patient's teeth. If done improperly, a mapping can result in significant distortions which can make the mapping of limited utility for teeth alignment/positioning.

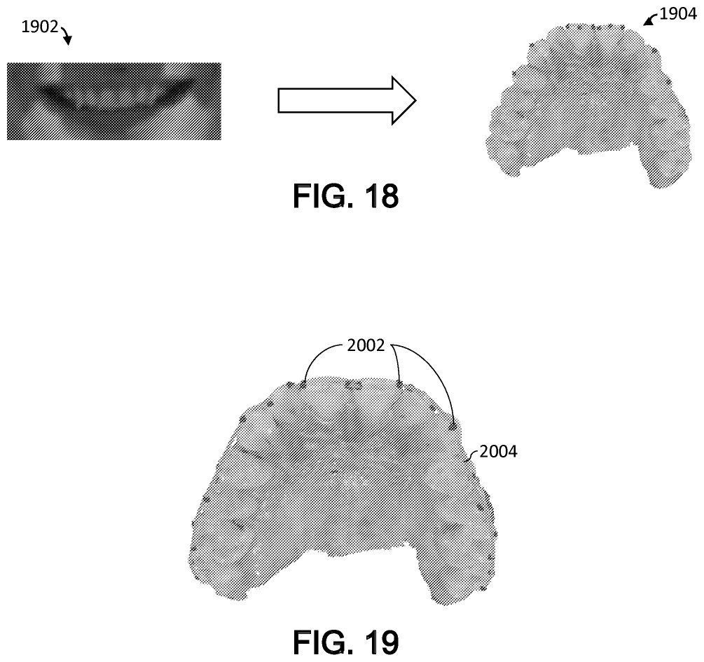

At block 1702, a system may be configured to project 2D control points that define the expected smile (e.g., the aesthetic arc depicted in FIG. 22) into 3D space. Similarly, anatomical points on both 2D images and 3D captures may be mapped to each other. FIG. 18 depicts an example of mapping anatomical points between a 2D projection 1802 and a 3D capture 1804 of a patient's teeth. Mapping between a 2D facial image and a 3D capture of the patient's teeth can be approached as a Perspective-n-Point problem given a set of n points in 3D space and their corresponding 2D projections. Given a set of known 3D points and their 2D projections, it may be possible to determine the camera pose (i.e., roll, pitch, yaw, and translations along the three orthogonal axes). In determining projections between a 2D image and 3D space, intrinsic parameters of the camera may be used (for example, focal length). For example, a transformation may be performed according to the formula sp_{c}=KRp_{w}, where s is a scaling factor, p_{c} is a 2D point, K is a matrix of the intrinsic camera parameters, R is a matrix of a desired rotation (extrinsic parameters), and p_{c} is the point in 3D space. In some embodiments, the rotation matrix R may instead be a translation matrix T, or the transformation between 2D and 3D space may include both a translation matrix T and a rotation matrix R.

In some cases, projecting from a 2D image to 3D space may be complex due to the lack of information in a 2D image about the third dimension (e.g., depth). In some embodiments, stereo vision may be used to aid in mapping a 2D image to a 3D space. For example, two cameras may be placed with some separation between them, and the images may be compared to determine depth information.

In some embodiments, a system may not project from 2D to 3D. This can mean that, for example, a curve displayed on a user interface to indicate an aesthetic arc may not be the same as an aesthetic curve determined for diagnostic and/or treatment purposes. In some embodiments, a user of the system may be allowed to move control points vertically because there is little change in depth along the vertical axis. However, the user may not be able to adjust control points in the horizontal direction because even small changes in horizontal position can correspond to large changes in depth. For example, returning to FIG. 16, a user of the system may move the three control points up and down to alter the aesthetic arc, but may not be able to move the control points horizontally. It will be appreciated that such limitations may not exist when a user is working with control points that are defined on a 3D scene.

At block 1704, an initial 3D curve may be created by passing through a series of points on the outward-facing surfaces of the maxillary 3D mesh. In some embodiments, a spline fitting may be used to produce a smooth curve through the points. In some embodiments, a B-spline algorithm may be used to calculate a 3D spline representing the dental arches. In some embodiments, a standard 3D curve may be selected from one or more template 3D splines that represent the dental arches. A template spline may be advantageous in some circumstances, such as when a shape memory alloy wire is used to move the teeth. In some embodiments an aesthetic arc can be a preformed arc that is selected from a catalog or database of aesthetic arcs. The preformed aesthetic arc can, in some embodiments, be used in calculating a double helix. While traditional approaches may consider only the aesthetic arc, the use of the double helix as described in this disclosure can enable the optimization of the orientation, inclination, etc. of the teeth, which can be difficult or even not possible when an aesthetic arc is considered in isolation. Such optimizations can improve functionality, reduce premature wear, and so forth. In some embodiments, an initial 3D curve may consider only the patient's upper maxillary teeth. FIG. 19 depicts an example of anatomical points 1902 to which an initial curve 1904 has been fitted by the system.

At block 1706, a user of the system may distort the initial 3D curve by, for example, moving one or more control points using a user interface, similar to how a user may modify the aesthetic arc in FIG. 16 by moving the control points. The system may calculate a distorted 3D curve from the moved control points. In some embodiments, the system may calculate distortions arising from the movement of each control point separately, and the individual distortions may be combined to determine an overall distortion of the initial 3D curve. In some embodiments, the distortion of each tooth may vary based upon the distance from a moved control point. For example, the distortion of a point may be weighted according to the distance from the point to the control point. In some embodiments, the distortion of a point may be calculated as sin²((1−d_{i,cp})/dₘₐₓ*π/2), where d_{i,cp} is the distance between an ith point of the curve and a control point and dₘₐₓ is the maximum distance between a point on the curve and the control point. In some embodiments, the distances are straight line distances between points.

At block 1708, the system may be configured to move anatomical points in accordance with the distorted 3D curve. For example, a system may determine the closest point on the initial 3D curve to each anatomical point, and the anatomical point may be distorted based on the distortion of the nearest point on the initial 3D curve. The anatomical points may be, for example, points along surfaces of the teeth.

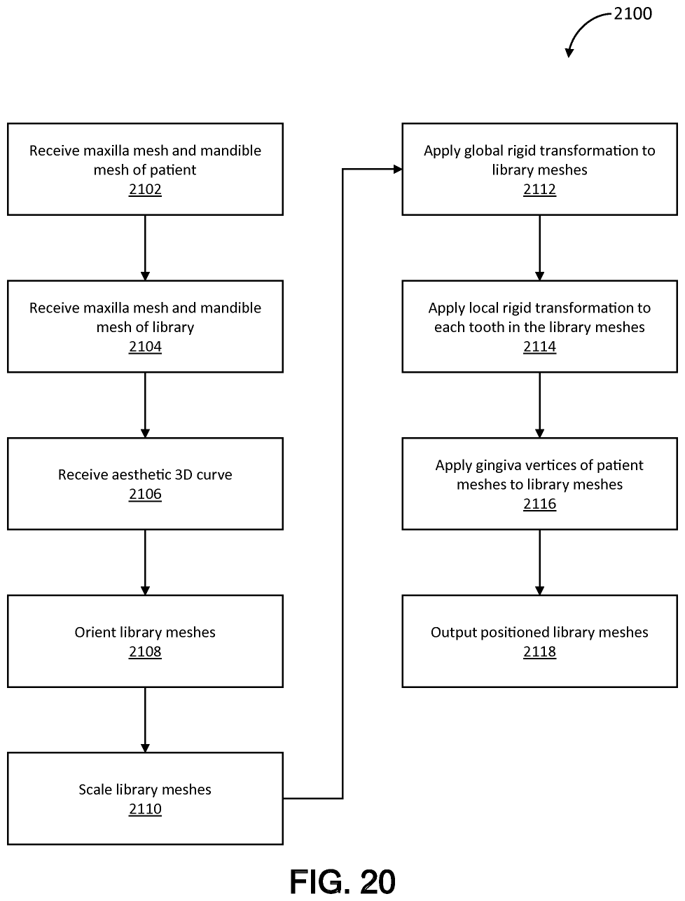

After determining an aesthetic 3D curve, the patient's teeth or artificial teeth may be positioned. FIG. 20 depicts an example process 2000 for positioning a library according to some embodiments. The library may be, for example, a library of artificial teeth or may be the patient's own teeth. Such libraries can be used in some embodiments for prosthetic and/or orthodontic treatment. A computer system may be configured to execute the process 2000. In some embodiments, a process can include fewer or additional steps. At block 2002, the system may receive a maxilla mesh and a mandible mesh of the patient. At block 2004, the system may receive a maxilla mesh and mandible mesh of a library, which may be, for example, the patient's own teeth or artificial teeth. At block 2006, the system may receive an aesthetic 3D curve, such as a curve produced according to the process 1700. At block 2008, the system may orient the library meshes and, at block 2010, may scale the library meshes in one or more dimensions to fit the patient. At block 2012, the system may apply a global rigid transformation to the library meshes, for example to align the library meshes to the patient by performing a translation in one or more directions, a rotation in one or more directions, or by performing rotations and translations in one or more directions. At block 2014, the system may apply local rigid transformations to each tooth (e.g., to one tooth, to multiple teeth, or to all teeth, either independently or in groupings of teeth) in the library meshes. At block 2016, the system may optionally apply gingiva vertices from the patient meshes to the library meshes. At block 2018, the system may output positioned library meshes, for example library meshes in which the teeth of the library have been globally and locally manipulated to better conform to the patient and the aesthetic 3D curve.

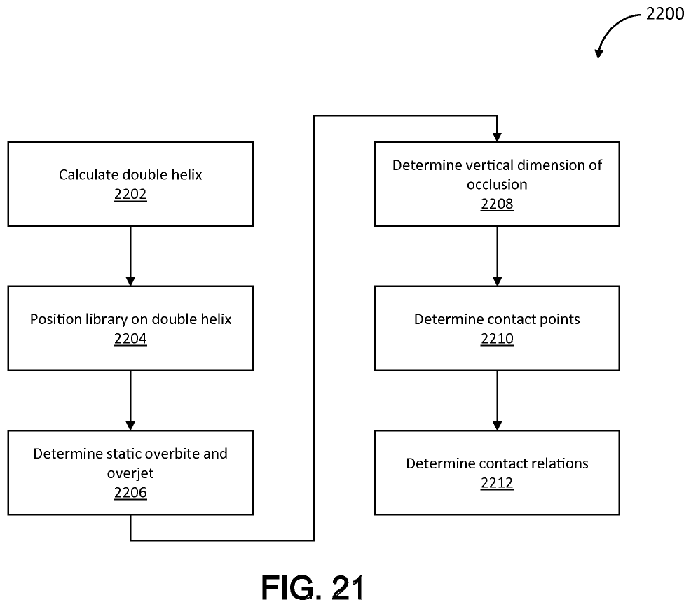

Initial fitting according to the aesthetic 3D curve may not result in ideal positioning of the teeth. Accordingly, FIG. 21 depicts an example process 2100 for optimizing the static positioning of a patient's teeth according to some embodiments which may be run on a computer system. At block 2102, a double helix may be computed, and at block 2104, the teeth may be positioned on the double helix. The double helix may be computed at least in part by determining various arcs along the outer edges, inner edges, or other anatomically relevant parts of the patient's teeth. For example, the system may determine a centering arc and a fitting arc. At block 2106, the system can determine static overbite and overjet. At block 2108, the system can determine a vertical dimension of occlusion. At block 2110 and block 2112, the system can determine contact points and contact relations, respectively. The steps in FIG. 21 can be performed in a different order. More or fewer steps may be included a in a process consistent with this disclosure.

### Double Helix Determination and Positioning

In some embodiments, an initial double helix can be based on the information about the patient, such as captured data about the positioning of the patient's teeth. For example, data about the patient's teeth can be used to generate an initial aesthetic arc. In some embodiments, the data can have metadata associated therewith. For example, the metadata may indicate buccal surfaces of the patient's teeth, which can be used for forming the aesthetic line.

In some embodiments, the initial aesthetic arc can be used for diagnosis, for developing a treatment plan, and so forth. A second double helix can be calculated based at least in part on an aesthetic line, which can be, for example, a random line, a manual design, or a line that is calculated automatically, for example based on facial scan data, pictures, etc.

In some embodiments, a zone of confrontation can be described by a geometric shape (e.g., a surface) such as, for example, a double helix. In some embodiments, the geometric shape can facilitate the positioning, modification, or both of one or more teeth. In some embodiments, the geometric shape can be modeled based at least in part on recorded data that is specific to a patient. In some embodiments, the geometric shape can be based on manipulated patient data, for example data that has been manipulated to achieve a desired aesthetic outcome, functional outcome, or both.

In some embodiments, patient-specific data may relate to, for example, one or more reference planes of the patient's skull, such as an axio-orbital plane, condylar slopes, and so forth. In some embodiments, patient-specific data can include photographs, facial scans, radiographs (e.g., lateral radiographs), CBCT images, and so forth.

In some embodiments, the geometric shape can be determined at least in part by an occlusal cap. Data related to an occlusal cap can include data related to the rear parts, the sagittal plane, or both. In some embodiments, the geometric shape data can define the architecture of the upper arch, the morphology of the upper teeth, or both. In some embodiments, the geometric shape for the upper arch, upper teeth, or both can impact the lower arch, may be complementary with the occlusal cap, or both. In some cases, the occlusal cap can be defined for the mandibular teeth. The occlusal cap can be a shape that includes the Curve of Spee and Wilson Curve. Calculation of the occlusal cap can take into account the condylar points, incisal points, and points of the distal lobes of the canines. Additional details can be found in, for example, U.S. Pat. No. 9,922,454 B2, titled “METHOD FOR DESIGNING AN ORTHODONTIC APPLIANCE,” the contents of which are incorporated by reference herein in their entirety.

In some cases, a patient may be edentulous, and the geometric shape can define a plate or surface on which teeth may be best applied according to, for example, metadata of the teeth (e.g., metadata of artificial teeth).

In some embodiments, determining a geometric shape can include constructing one or more arcs. For example, in some embodiments, any combination of one or more of an aesthetic arc, a centering arc, and a fitting arc, as described herein, can be used for determining the geometric shape. In some embodiments, one or more the arcs may have been previously determined, for example as described above.

As discussed above, in some embodiments a helical structure can be calculated, and teeth (e.g., the patient's own teeth, artificial teeth, or both) can be fitted to the helical structure. The helical structure can be defined, as discussed above, at least in part by the aesthetic arc. In some embodiments, additional structural data about the patient can be used in calculating the double helix.

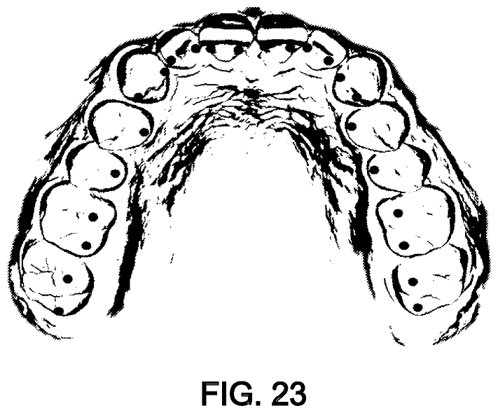

In some embodiments, a centering arc, a fitting arc, or both can be used in combination with the aesthetic arc to define a double helix. FIG. 22 shows an example of points for defining an aesthetic arc for maxillary teeth according to some embodiments. In some embodiments, the aesthetic arc can be defined at least in part by the incisive edges, canine edges, buccal cusps of the premolars, and/or buccal cusps of the molars. In some embodiments, a centering arc can be an arc that describes the centers or other points on the surfaces of the teeth. An example of points defining a centering arc for maxillary teeth is shown in FIG. 23. The centering arc can pass through, for example, palatal cusps, mesial ridges, and/or distal ridges of the incisors and/or canines. A fitting arc for maxillary teeth can describe an interior boundary of the maxillary teeth, as shown in FIG. 24. In some embodiments, the fitting arc can be determined from the marginal ridges of the incisors, canines, premolars, and molars. In some embodiments, the fitting arc can consider molar pits (e.g., the fitting arc can be aligned with the molar pits). In some embodiments, a fitting arc can be determined from the incisive edges, canine tips, vestibular cusps of the premolars and molars, or any combination of these features. It will be appreciated that different arcs can be used for similar purposes, although arcs preferably relate to structures of the teeth so that the arcs are anatomically relevant and have a consistent, logical structure.

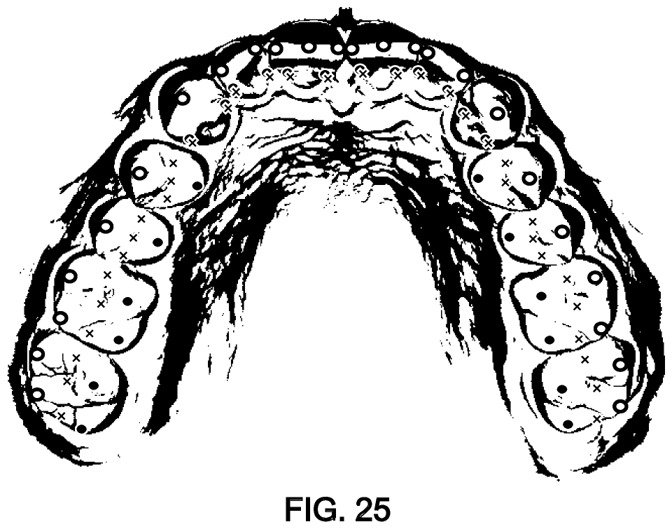

FIG. 25 is an example illustration showing a centering arc (white circles with black outline), centering arc (black circles with white outline), and fitting arc (black crosses with white outline) for maxillary teeth.

In some embodiments, an arc or arcs can be used to define at least in part the shape, positioning, or both of the teeth. As an example, the three arcs of a cuspid tooth can form an inverted “V” shape in an anterior or posterior view. In some embodiments, the arcs can consider one or more future locations of one or more teeth. In some embodiments, the arcs can be determined by considering segments individually, although this is not necessary. In some cases, segments can be considered in groups or as a whole when determining an arc.

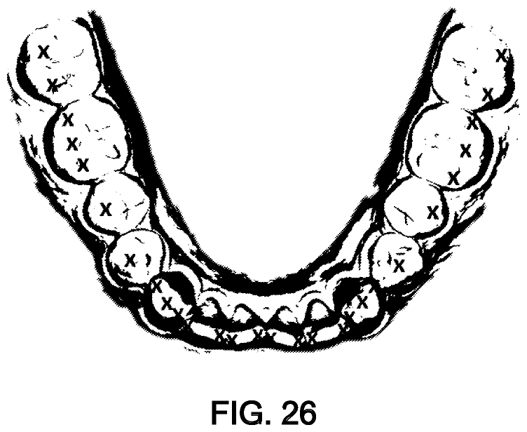

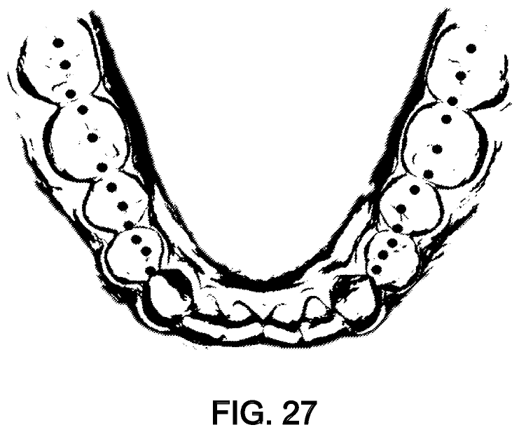

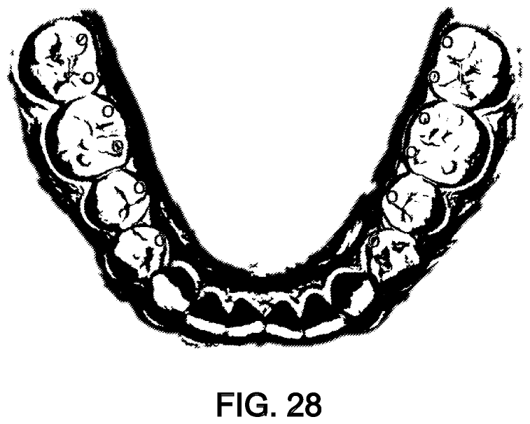

In some embodiments, arcs can be determined for the maxillary teeth, for example as described above. In some embodiments, arcs can be determined for mandibular teeth. FIGS. 26-29 illustrate example arcs for mandibular teeth. FIG. 26 illustrates a fitting arc for the mandibular teeth according to some embodiments. As shown in FIG. 26, a fitting arc can pass through incisive and canine edges, buccal cusps of the premolars, and/or buccal cusps of the molars. FIG. 27 illustrates a centering arc for mandibular teeth according to some embodiments. FIG. 28 illustrates a guiding arc for mandibular teeth according to some embodiments. The guiding arc can pass through, for example, the lingual cusps of the premolars and molars.

In some embodiments, if a patient is edentulous or if the existing teeth have large deviations from a desired placement, segmentation, identification of points, and so forth may be done partially or wholly manually. For example, an AI/ML model may fail to identify relevant features when a patient either is edentulous or when teeth deviate too significantly from expected positions, orientations, or both. In some embodiments, segments can be orthogonal to one or more features, such as an aesthetic vestibular arc line. It is not, however, necessary that segments be orthogonal to an arc line. For example, some segments, such as the canine, may not be orthogonal to an aesthetic vestibular arc line. In some embodiments, segments can be created and can be separated from each other. Segments can have distances that correspond to the average lengths of teeth. For example, a segment for a molar can have a distance or depth of about 8 mm.

In some embodiments, if a patient has sufficient teeth such as, for example, all teeth, substantially all teeth, a majority of teeth, or a minority of teeth, a metadata point projection can be made on the aesthetic arc line. In some embodiments, a metadata point projection can be made using AI/ML models. In some embodiments, a guide segment of length x and angle α can be formed by the guide segment with respect to the axio-orbital plane can be modeled. The guide segment can be from the aesthetic arc to the fitting arc, and the distance x can be a distance from the aesthetic arc to the fitting arc for a particular tooth. In some embodiments, particular distances and angles can be associated with different types of teeth, for example as indicated in the table below. The distances and angles can vary for different condylar slopes. For example, the table below can be for 50° condylar slope.

| Tooth Type | Inclination (α, °) | Distance (x, mm) |
|---|---|---|
| Medial Incisor | 57.2 ± 9.7 | 3.4 ± 0.8 |
| Lateral Incisor | 53.6 ± 10.5 | 3.1 ± 0.9 |
| Canine | 47.7 ± 8.1 | 3.5 ± 1.1 |
| First Premolar | 30.7 ± 9.7 | 3.3 ± 0.4 |
| Second Premolar | 20.7 ± 9.7 | 3.2 ± 0.6 |
| First Molar | 12.0 ± 2.7 | 3.4 ± 0.8 |
| Second Molar | 8.7 ± 9.7 | 2.7 ± 0.9 |

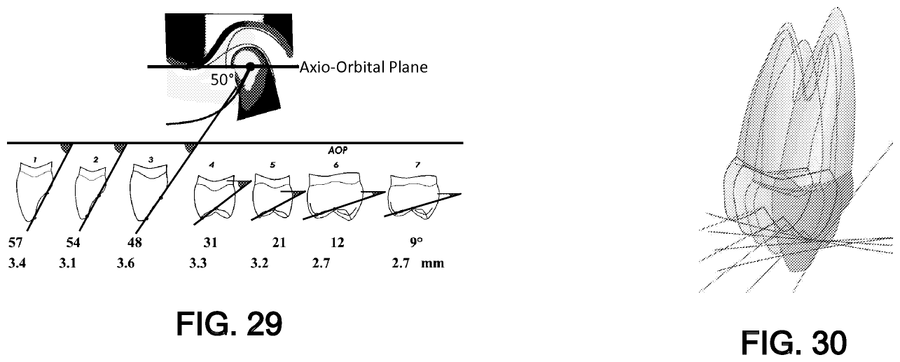

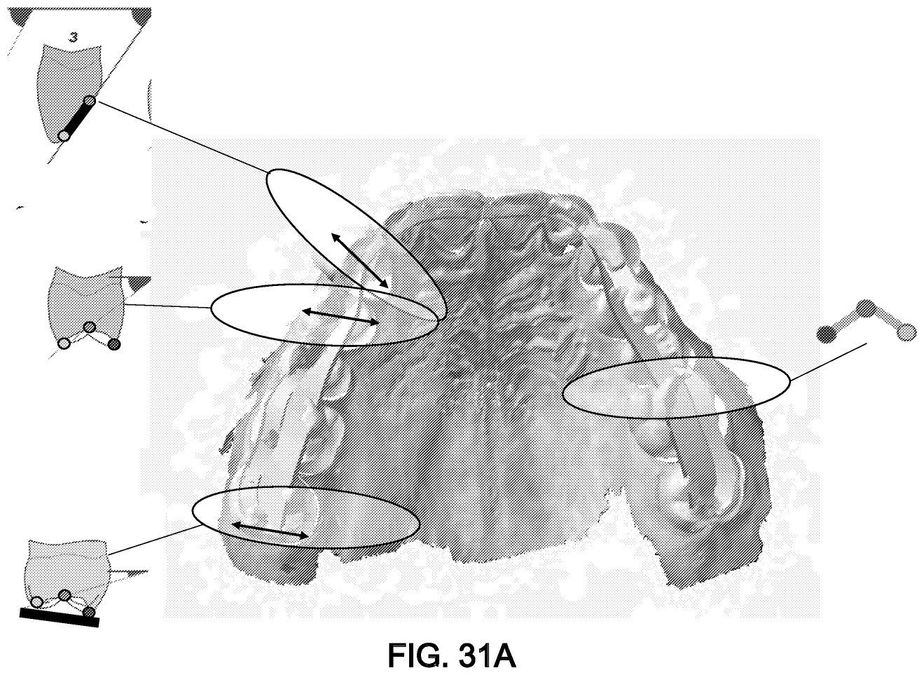

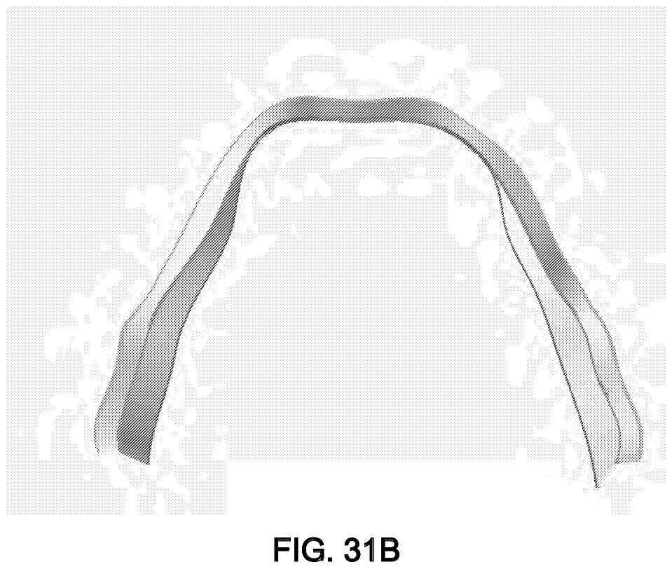

FIG. 29 is an example illustration of the slopes and distances for different teeth, corresponding to the table above. As illustrated in FIG. 30, teeth can have sequential slopes. FIG. 31A shows the inclination angles associated with various teeth in the mouth of a patient. FIG. 31B shows a double helix according to some embodiments.

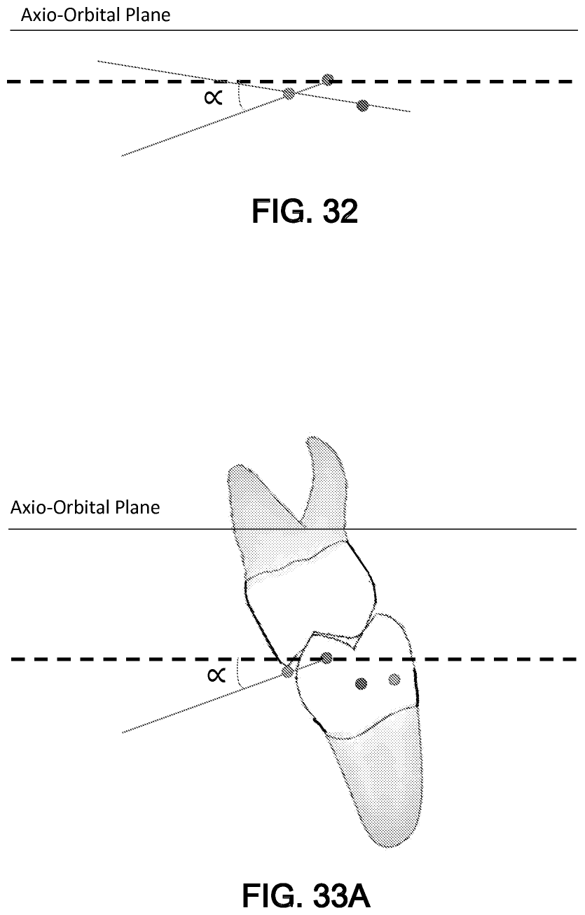

FIG. 32 is a cross-section view showing the inclination a with respect to an axio-orbital plane for an example tooth (e.g., a molar).

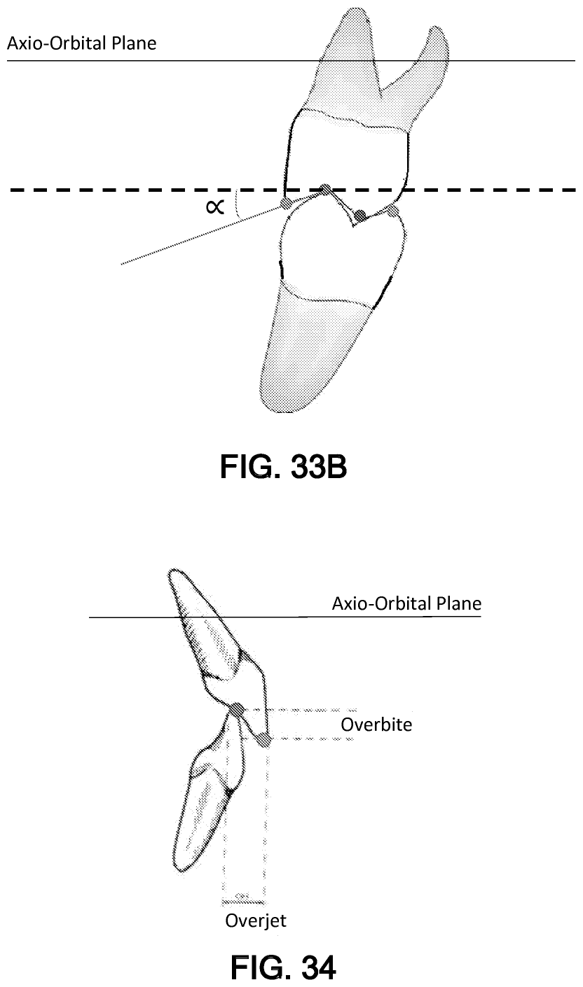

FIGS. 33A and 34B illustrate an example of fitting maxillary and mandibular teeth. As shown in FIG. 33A, an aesthetic arc position can be frozen in place for a maxillary tooth. In FIG. 33B, the tooth has been rotated to achieve a desired inclination angle α. A corresponding mandibular tooth can be manipulated to maintain proper alignment with the positioned maxillary tooth.

In some embodiments, a double helix can be formed at least in part by obtaining an external arc (e.g., an aesthetic arc) that can define an external limit of the double helix; creating, for each segment, from a projection point, a segment of length x and angle α that may correspond to, for example, a tooth; defining one or more intermediate points at the end of the segment; and determining, for each segment, an innermost point which may be based at least in part on statistical data representing average tooth width, a projection of a corresponding tooth metadata point, or both. In some embodiments, an AI/ML model can be used to determine one or more points to form a double helix structure. In some embodiments, a practitioner may make manual adjustments to the double helix.

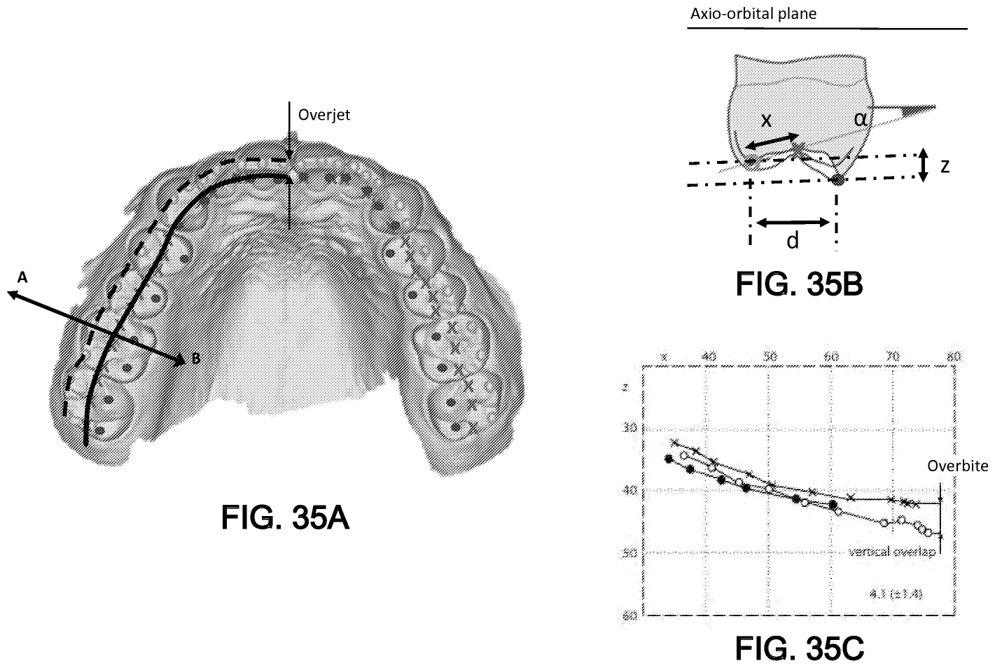

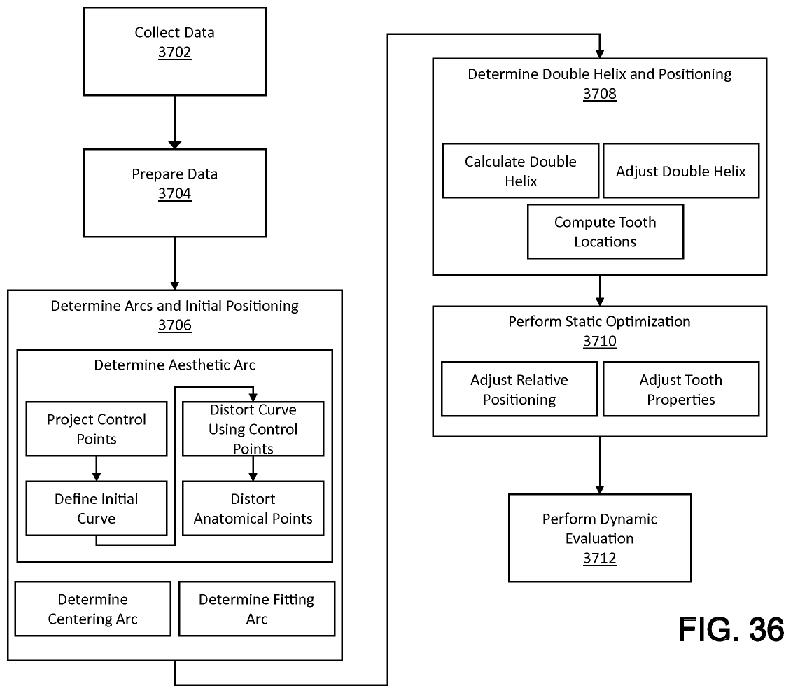

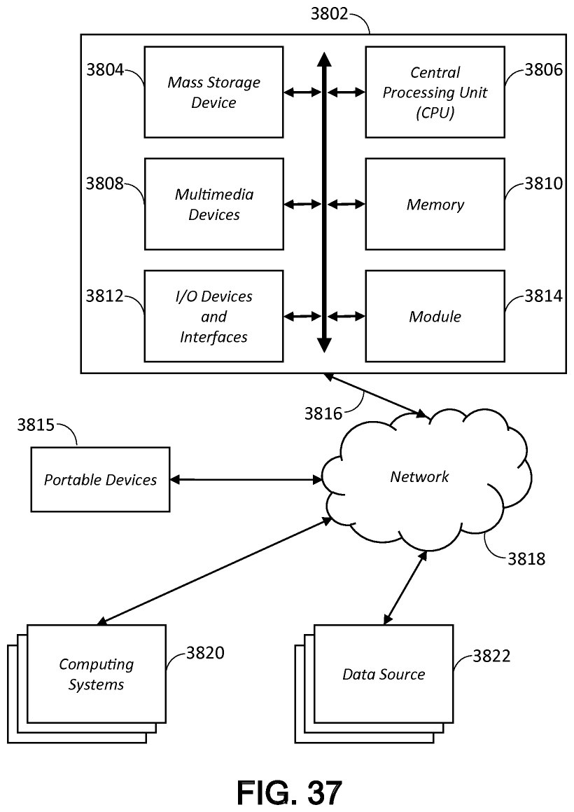

FIGS. 35A-36C illustrate relationships between the aesthetic arc, fitting arc, centering arc, overbite, and overjet. FIG. 35A shows a view of maxillary teeth with an aesthetic arc (dashed line, open circles), fitting arc (solid line, crosses), and centering arc (solid circles). FIG. 35B shows a cross-section across the segment AB in FIG. 35A. As shown in FIG. 35B, there can be a distance x between the aesthetic arc and the fitting arc, a distance d between the aesthetic arc and the centering arc, and a vertical distance z between the aesthetic arc and the centering arc. Returning to FIG. 35A, the distance x at the incisors can define an overjet value. FIG. 35C shows that that value z can define an overbite value when measured at the incisors.

In some embodiments, a fitting arc can be determined by a system using a table such as the table of above. For example, after the aesthetic arc is determined, a fitting arc can be constructed, the points of the fitting arc having distances from corresponding points on the aesthetic arc (e.g., buccal points), for example as defined in the table above or in a similar table. The fitting arc can be positioned relative to the aesthetic arc such that a line segment drawn between a point on the aesthetic arc and a point on the fitting arc has an angle with respect to the axio-orbital plane as indicated above.

In some embodiments, a centering arc can be determined based at least in part on the aesthetic and/or fitting arcs. In some embodiments, for a premolar and/or molar, the centering arc can be at an average distance d of about 6 mm from the corresponding buccal point. For anterior teeth (e.g., canines and incisors), the centering arc can correspond to an overjet of about 4 mm. In some embodiments, the aforementioned distances can be modified manually, automatically, or semi-automatically depending on the patient and the treatment needs. A centering point can be higher or lower, or closer or further away from the axio-orbital plane by a distance z, depending on anthropomorphic values. For example, a mesio-palatal cusp of tooth 26 can be 0.8 mm lower than the point on the aesthetic arc corresponding to tooth 26. Example z positions of anatomical points corresponding the centering arc with respect to the aesthetic arc are given in table below, wherein positive values indicate that the point characterizing the centering arc is below the corresponding point defining the aesthetic arc.

| Centering Arc Point | Aesthetic Arc Point | z (mm) |
|---|---|---|
| Distal buccal cusp | Distal palatal cusp | 0.3 |
| of second molar | of second molar | |
| Mesial buccal cusp | Mesial palatal cusp | 0.9 |
| of second molar | of second molar | |
| Distal buccal cusp | Distal palatal cusp | 0.2 |
| of first molar | of first molar | |
| Mesial buccal cusp | Mesial palatal cusp | 0.8 |
| of first molar | of first molar | |
| Buccal cusp of | Palatal cusp of | 0 |
| second premolar | second premolar | |
| Buccal cusp of | Palatal cusp of | −0.7 |
| first premolar | first premolar | |

FIG. 34 shows an example of incisor alignment according to some embodiments. In some embodiments, an incisal edge along the aesthetic arc can be positioned, and the incisor can be angled to obtain the desired inclination angle. The mandibular incisor can then be placed to have a desired overbite, which can determine at least in part the vertical positioning of the incisors. The mandibular incisor can then be oriented to preserve a desired overjet.

In some embodiments, the calculation of the double helix can include the occlusal cap. The occlusal cap can include the Curve of Spee, which defines the curvature of the mandibular occlusal plane, starting from the edge of the mandibular incisor and extending to the condyle. In some embodiments, the buccal cusps of the mandibular cuspid teeth can be manipulated to conform to the Curve of Spee, which can constrain the overall fitting of the teeth. In some embodiments, the location of the incisal edge and the condylar points can be fixed and the remaining positions can be adjusted by altering the concavity so that the occlusal surfaces of the maxillary and mandibular first molars are in alignment.

### Static Optimization

A system may be configured to perform static optimization on the mandibular and maxillary libraries. This may be done before or after positioning the libraries, although it may be advantageous to perform static optimization after alignment. With reference again to FIG. 20, at block 2106, a system may determine an optimal overbite and overjet. The system may be configured to take initial positioning values of the teeth and calculate a transformation to apply to achieve desired overjet and overbite values. To obtain the desired overbite, overjet, or both, an algorithm can be configured to move the mandible. The movement of the mandible can be based on simulated motion data or on registered motion of the patient's jaw. The system may be configured to determine an overbite value by computing the average vertical difference between the incisal edges of the maxillary and mandibular teeth. Similarly, the system may compute an overjet value by determining the average horizontal difference between maxillary and mandibular incisal edges.

At block 2108, the system may determine an optimal vertical dimension of occlusion. Given an optimal overbite value, optimal overjet value, positioned libraries (e.g., maxillary and mandibular meshes), and a centric relation, the system may determine a mandibular mesh transformation to apply to achieve an optimal vertical dimension of occlusion. For example, the system may find a frame in a capture of the patient's jaw movement that corresponds to an optimal overbite and/or overjet value. The system may transform the mandibular mesh to the optimal overbite and/or overjet position. In some cases, the system may determine that the overbite and/or overjet is acceptable and may not select a new position, while in other embodiments, the overbite and/or overjet may be changed to increase or decrease the overbite and/or overjet. The overbite and overjet analysis may be performed when a mandibular library is placed or may be done after positioning the mandibular teeth. In some cases, it may be advantageous to perform the overbite and overjet analysis when the mandibular library is placed, such as in a prosthetic workflow. In other circumstances, it may be preferable to perform the overbite and overjet analysis after positioning the mandibular teeth.

### Dynamic Evaluation

In some embodiments, dynamics can be considered. For example, in some embodiments, motion with respect to a reference such as to the axio-orbital plane can be considered. In some embodiments, dynamics information can come from a patient's movements. In some embodiments, dynamics information can come from simulated movements. In some embodiments, simulation of a patient's movements can be performed by modeling movement around the posterior condylar points.

It is important that functionality (e.g., speaking, eating, etc.) be preserved and that positioning of the teeth does not result in uneven or premature wearing down of surfaces of the teeth. For example, it is important that surfaces of the teeth are aligned so that functionality (for example, chewing) is not compromised and that the positioning of the teeth be suitable throughout the patient's range of movement. Accordingly, it is advantageous to determine the contacts between maxillary and mandibular teeth. At block 2110, a system may determine the contact points from the maxilla mesh, mandible mesh, and a capture of the movements of the patient's jaw. For each frame in the animation or for a subset of frames in the capture of the patient's movements, the system may determine contact points between the teeth. For example, the methods described in U.S. Pat. No. 10,582,992, the entire contents of which are incorporated by reference herein in their entirety and for all purposes, may be used.

In some embodiments, the quantity of frames in the capture of the patient's movements may be reduced in order to speed up the process of calculating contact points. For example, frames may be discarded if the maxillary and mandibular meshes are too far apart. For example, if the distance between the central vertex of the maxilla and the central vertex of the mandible is greater than a threshold value, the frame may be discarded. For example, frames may be discarded if the distance is greater than about 5 mm, greater than about 8.5 mm, greater than about 10 mm, or any other greater or lesser separation as may be desirable for reducing the quantity of frames while preserving sufficient information.

In some embodiments, frames may be discarded if the movement from one frame to another is below a threshold value. For example, if the distance between the central vertices of the maxillary and mandibular meshes changes by less than about 0.005 mm, then at least one of the frames may be discarded. In some embodiments, the data set may be further reduced by, for example, taking only a fraction of the remaining frames. For example, in some embodiments, the system may keep one out of every eight frames, one out of every ten frames, and so forth. The system may then calculate contact points between the mandibular and maxillary meshes from the reduced data set.

The calculation of the double helix at block 2102 can lead to the repositioning of the upper teeth at block 2104. The repositioning of the upper teeth can enable the determination of overbite and/or overjet at block 2106 by repositioning the lower incisors relative to the upper incisors. At block 2108, the vertical dimension of occlusion (VDO) can be determined in relation to the overbite. After the positioning of the teeth and the VDO are modified, a system can be used to automatically, semi-automatically, or manually tune the positioning (e.g., orientation) and shape of teeth to obtain optimal contact in a static state at block 2110. Advantageously, the system may then enable optimization of functional tooth positioning.

At block 2112, the system may compute the contact relations of the maxillary and mandibular meshes based on the maxillary mesh, the mandibular mesh, the capture of the patient's movements, the contact points, and the semantic segmentations of the maxillary and mandibular meshes. The system may, for each animation frame with contact points (e.g., the frames that were kept at block 2110), determine contact vertices in the maxilla and mandible for each point where a tooth in the mandibular mesh contacts a tooth in the maxillary mesh. The system may compute one or more distances for each contact point and may store the information in a table, database, spreadsheet, array, and so forth. The system may compute the contact relations between each unique pair of teeth over successive frames. The system may track the evolution of the separation between each unique pair of teeth over time by calculating, for one or more frames, the minimal distance between the two closest pixels (one on each tooth) of each unique pair of teeth. A contact relation may, alternatively or additionally, be characterized by a single minimal distance between two teeth.

FIG. 36 is a flow chart that illustrates an overview of an example process for planning an orthodontic and/or prosthodontic procedure consistent with this disclosure. At block 3602, a system can collect data such as dental impressions, facial scans, portraits, and so forth that can be used in treatment planning. At block 3604, the system can be configured to prepare the patient data, which can include performing transformations on the data or otherwise modifying the data for use in treatment planning. At block 3606, the system can determine one or more arcs and initial positioning of the teeth. As shown in FIG. 36, the system can, at block 3606, determine an aesthetic arc, which can include projecting control points, defining an initial curve, distorting the curve using the control points, and distorting anatomical points to fit to the distorted curve. The system can determine a centering arc and/or a fitting arc, which can be related to the aesthetic arc, anatomical points on the patient's teeth, and so forth. At block 3608, the system can, based at least in part on the determined arcs, calculate a double helix structure, adjust the double helix (which can be manual, automatic, or semi-automatic), and compute tooth locations. At block 3610, the system can perform static optimization, which can include adjusting the relative positioning of the teeth and/or adjusted various properties of the teeth. For example, static optimization can include altering the size, shape, or both of one or more prosthetic teeth. At block 3612, the system can perform dynamic evaluation as described above, which can include consideration of the contact relations between mandibular and maxillary teeth.

### Computer Systems

FIG. 38 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 38. The example computer system 3802 is in communication with one or more computing systems 3820 and/or one or more data sources 3822 via one or more networks 3818. While FIG. 38 illustrates an embodiment of a computing system 3802, it is recognized that the functionality provided for in the components and modules of computer system 3802 may be combined into fewer components and modules, or further separated into additional components and modules.

The computer system 3802 can comprise a module 3814 that carries out the functions, methods, acts, and/or processes described herein. The module 3814 is executed on the computer system 3802 by a central processing unit 3806 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 3802 includes one or more processing units (CPU) 3806, which may comprise a microprocessor. The computer system 3802 further includes a physical memory 3810, such as random-access memory (RAM) for temporary storage of information, a read only memory (ROM) for permanent storage of information, and a mass storage device 3804, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 3802 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 3802 includes one or more input/output (I/O) devices and interfaces 3812, such as a keyboard, mouse, touch pad, and printer. The I/O devices and interfaces 3812 can include one or more display devices, such as a monitor, that allows the visual presentation of data to a user. More particularly, a display device provides for the presentation of GUIs as application software data, and multi-media presentations, for example. The I/O devices and interfaces 3812 can also provide a communications interface to various external devices. The computer system 3802 may comprise one or more multi-media devices 3808, such as speakers, video cards, graphics accelerators, and microphones, for example.

The computer system 3802 may run on a variety of computing devices, such as a server, a Windows server, a Structure Query Language server, a Unix Server, a personal computer, a laptop computer, and so forth. In other embodiments, the computer system 3802 may run on a cluster computer system, a mainframe computer system and/or other computing system suitable for controlling and/or communicating with large databases, performing high volume transaction processing, and generating reports from large databases. The computing system 3802 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 3802 illustrated in FIG. 38 is coupled to a network 3818, such as a LAN, WAN, or the Internet via a communication link 3816 (wired, wireless, or a combination thereof). Network 3818 communicates with various computing devices and/or other electronic devices. Network 3818 is communicating with one or more computing systems 3820 and one or more data sources 3822. The module 3814 may access or may be accessed by computing systems 3820 and/or data sources 3822 through a web-enabled user access point. Connections may be a direct physical connection, a virtual connection, and other connection type. The web-enabled user access point may comprise a browser module that uses text, graphics, audio, video, and other media to present data and to allow interaction with data via the network 3818.

Access to the module 3814 of the computer system 3802 by computing systems 3820 and/or by data sources 3822 may be through a web-enabled user access point such as the computing systems' 3820 or data source's 3822 personal computer, cellular phone, smartphone, laptop, tablet computer, e-reader device, audio player, or another device capable of connecting to the network 3818. Such a device may have a browser module that is implemented as a module that uses text, graphics, audio, video, and other media to present data and to allow interaction with data via the network 3818.

The output module may be implemented as a combination of an all-points addressable display such as a cathode ray tube (CRT), a liquid crystal display (LCD), a plasma display, or other types and/or combinations of displays. The output module may be implemented to communicate with input devices 3812 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 3802 may comprise a physical or logical connection established between a remote microprocessor and a mainframe host computer for the express purpose of uploading, downloading, or viewing interactive data and databases online in real time. The remote microprocessor may be operated by an entity operating the computer system 3802, including the client server systems or the main server system, an/or may be operated by one or more of the data sources 3822 and/or one or more of the computing systems 3820. In some embodiments, terminal emulation software may be used on the microprocessor for participating in the micro-mainframe link.

In some embodiments, computing systems 3820 who are internal to an entity operating the computer system 3802 may access the module 3814 internally as an application or process run by the CPU 3806.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 3802 may include one or more internal and/or external data sources (for example, data sources 3822). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 3802 may also access one or more databases 3822. The databases 3822 may be stored in a database or data repository. The computer system 3802 may access the one or more databases 3822 through a network 3818 or may directly access the database or data repository through I/O devices and interfaces 3812. The data repository storing the one or more databases 3822 may reside within the computer system 3802.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

### Example Clauses

Examples of implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.

Clause 1. A computer-implemented method for dental treatment planning comprising: receiving, by a computing system, patient data associated with a patient; determining, by the computing system, at least one arc, the arc corresponding to anatomical points of teeth of a tooth library; determining, by the computing system based on the at least one arc, a double helix, the double helix to be used for fitting a tooth library; determining, by the computing system, positions of the teeth of the tooth library on the double helix; and optimizing, by the computing system, the teeth of the tooth library.

Clause 2. The method of Clause 1, wherein the patient data comprises tooth data.

Clause 3. The method of Clause 1, wherein the patient data comprises morphometric data.

Clause 4. The method of Clause 1, wherein determining at least one arc comprises: providing the patient data to an AI model, the AI model trained to identify anatomical points of the teeth of the tooth library.

Clause 5. The method of Clause 1, wherein determining a double helix comprises providing the at least one arc to an AI model configured to determine the double helix based at least in part on the at least one arc.

Clause 6. The method of clause 1, wherein optimizing the teeth of the tooth library comprises determining, using an AI model, a position, a rotation, or both of each tooth of the tooth library, the AI model configured to optimize functional and aesthetic positioning of the teeth.

Clause 7. The method of Clause 1, further comprising performing, by the computing system, dynamic evaluation of the positions of the teeth of the tooth library.

Clause 8. The method of Clause 1, wherein determining at least one arc comprises determining an aesthetic arc, and wherein determining the aesthetic arc comprises: projecting, by the computing system, one or more control points onto an image of the patient; defining, by the computing system, based at least in part on the one or more control points, an initial curve; determining a final curve by modifying, by the computing system, at least one control point; and determining, by the computing system, locations of one or more anatomical points based at least in part on the final curve, the locations of the one or more anatomical points defining at least in part the aesthetic arc.

Clause 9. The method of Clause 1, wherein determining the at least one arc comprises determining an aesthetic arc, a centering arc, and a fitting arc.

Clause 10. The method of Clause 9, wherein determining the least one arc further comprises determining a guiding arc associated with mandibular teeth.

Clause 11. The method of Clause 1, wherein optimizing the teeth of the tooth library comprises adjusting a relative positioning of one or more teeth in the tooth library.

Clause 12. The method of Clause 11, wherein adjusting the relative positioning comprises adjusting an overbite value and an overjet value.

Clause 13. The method of Clause 1, wherein optimizing the teeth of the tooth library comprises adjusting any combination of one or more of a size, shape, or rotation of at least one tooth of the tooth library.

Clause 14. The method of Clause 1, wherein the tooth library comprises a library of the patient's teeth, and wherein the method further comprises: identifying, by the computing system, one or more teeth of the tooth library; and annotating, by the computing system, one or more anatomical points of each tooth of the one or more teeth of the tooth library.

Clause 15. The method of Clause 1, wherein the tooth library comprises a library of artificial teeth, and wherein the method further comprises: selecting, by the computing system based at least in part on the captured patient data, a tooth library from a plurality of prosthetic tooth libraries.

Clause 16. The method of Clause 7, wherein optimizing the teeth of the tooth library comprises determining contact points between maxillary teeth of the patient and mandibular teeth of the patient, wherein performing dynamic evaluation comprises determining contact relations between the maxillary teeth of the patient and the mandibular teeth of the patient during movement of a jaw of the patient.

Clause 17. A system for dental treatment planning comprising: a computer readable storage medium having program instructions embodied therewith; and one or more processors configured to execute the program instructions to cause the system to: receive patient data associated with a patient; determine at least one arc, the arc corresponding to anatomical points of teeth of a tooth library; determine, based on the at least one arc, a double helix, the double helix to be used for fitting a tooth library; determine positions of the teeth of the tooth library on the double helix; and optimize the teeth of the tooth library.

Clause 18. The system of Clause 17, wherein the patient data comprises tooth data. Clause 19. The system of Clause 17, wherein the patient data comprises morphometric data.

Clause 20. The system of Clause 17, wherein determining at least one arc comprises: providing the patient data to an AI model, the AI model trained to identify anatomical points of the teeth of the tooth library.

Clause 21. The system of Clause 17, wherein determining a double helix comprises providing the at least one arc to an AI model configured to determine the double helix based at least in part on the at least one arc.

Clause 22. The system of clause 17, wherein optimizing the teeth of the tooth library comprises determining, using an AI model, a position, a rotation, or both of each tooth of the tooth library, the AI model configured to optimize functional and aesthetic positioning of the teeth.

Clause 23. The system of Clause 17, wherein the computer readable storage medium has instructions embodied therewith that, when executed by the one or more processors, cause the system to: perform dynamic evaluation of the positions of the teeth of the tooth library.

Clause 24. The system of Clause 17, wherein determining at least one arc comprises determining an aesthetic arc, and wherein determining the aesthetic arc comprises: project one or more control points onto an image of the patient; define based at least in part on the one or more control points, an initial curve; define a final curve by modifying at least one control point of the one or more control points; and determine locations of one or more anatomical points based at least in part on the final curve, the locations of the one or more anatomical points defining at least in part the aesthetic arc.

Clause 25. The system of Clause 17, wherein determining the at least one arc comprises determining an aesthetic arc, a centering arc, and a fitting arc.

Clause 26. The system of Clause 25, wherein determining the at least one arc further comprising determining a guiding arc associated with mandibular teeth.

Clause 27. The system of Clause 17, wherein optimizing the teeth of the tooth library comprises adjusting a relative positioning of one or more teeth in the tooth library.

Clause 28. The system of Clause 27, wherein adjusting the relative positioning comprises adjusting an overbite value and an overjet value.

Clause 29. The system of Clause 17, wherein optimizing the teeth of the tooth library comprises adjusting any combination of one or more of a size, shape, or rotation of at least one tooth of the tooth library.

Clause 30. The system of Clause 17, wherein the tooth library comprises a library of the patient's teeth, and wherein the computer readable storage medium has instructions embodied therewith that, when executed by the one or more processors, cause the system to: identify one or more teeth of the tooth library; and annotate one or more anatomical points of each tooth of the one or more teeth of the tooth library.

Clause 31. The system of Clause 17, wherein the tooth library comprises a library of artificial teeth, and wherein the computer readable storage medium has instructions embodied therewith that, when executed by the one or more processors, cause the system to: select, based at least in part on the patient data, a tooth library from a plurality of prosthetic tooth libraries.

Clause 32. The system of Clause 23, wherein optimizing the teeth of the tooth library comprises determining contact points between maxillary teeth of the patient and mandibular teeth of the patient, wherein performing dynamic evaluation comprises determining contact relations between the maxillary teeth of the patient and the mandibular teeth of the patient during movement of a jaw of the patient.

1. A computer-implemented method for dental treatment planning comprising:
receiving, by a computing system, patient data associated with a patient;
determining, by the computing system, at least one arc, the arc corresponding to anatomical points of teeth of a tooth library;
determining, by the computing system based on the at least one arc, a double helix, the double helix to be used for fitting the tooth library;
determining, by the computing system, positions of the teeth of the tooth library on the double helix; and
optimizing, by the computing system, the teeth of the tooth library.

2. The method of claim 1, wherein the patient data comprises tooth data.

3. The method of claim 1, wherein the patient data comprises morphometric data.

4. The method of claim 1, wherein determining the at least one arc comprises:
providing the patient data to an AI model, the AI model trained to identify the anatomical points of the teeth of the tooth library.

5. The method of claim 1, wherein determining the double helix comprises providing the at least one arc to an AI model configured to determine the double helix based at least in part on the at least one arc.

6. The method of claim 1, wherein optimizing the teeth of the tooth library comprises determining, using an AI model, a position, a rotation, or both of each tooth of the tooth library, the AI model configured to optimize functional and aesthetic positioning of the teeth.

7. The method of claim 1, further comprising performing, by the computing system, dynamic evaluation of the positions of the teeth of the tooth library.

8. The method of claim 1, wherein determining the at least one arc comprises determining an aesthetic arc, and wherein determining the aesthetic arc comprises:
projecting, by the computing system, one or more control points onto an image of the patient;
defining, by the computing system, based at least in part on the one or more control points, an initial curve;
determining a final curve by modifying, by the computing system, at least one control point; and
determining, by the computing system, locations of one or more anatomical points based at least in part on the final curve, the locations of the one or more anatomical points defining at least in part the aesthetic arc.

9. The method of claim 1, wherein determining the at least one arc comprises determining an aesthetic arc, a centering arc, and a fitting arc.

10. The method of claim 9, wherein determining the at least one arc further comprises determining a guiding arc associated with mandibular teeth.

11. The method of claim 1, wherein optimizing the teeth of the tooth library comprises adjusting a relative positioning of one or more teeth in the tooth library.

12. The method of claim 11, wherein adjusting the relative positioning comprises adjusting an overbite value and an overjet value.

13. The method of claim 1, wherein optimizing the teeth of the tooth library comprises adjusting any combination of one or more of a size, shape, or rotation of at least one tooth of the tooth library.

14. The method of claim 1, wherein the tooth library comprises a library of the patient's teeth, and wherein the method further comprises:
identifying, by the computing system, one or more teeth of the tooth library; and
annotating, by the computing system, one or more anatomical points of each tooth of the one or more teeth of the tooth library.

15. The method of claim 1, wherein the tooth library comprises a library of artificial teeth, and wherein the method further comprises:
selecting, by the computing system based at least in part on the patient data, the tooth library from a plurality of prosthetic tooth libraries.

16. The method of claim 7, wherein optimizing the teeth of the tooth library comprises determining contact points between maxillary teeth of the patient and mandibular teeth of the patient, wherein performing the dynamic evaluation comprises determining contact relations between the maxillary teeth of the patient and the mandibular teeth of the patient during movement of a jaw of the patient.

17. A system for dental treatment planning comprising:
a computer readable storage medium having program instructions embodied therewith; and
one or more processors configured to execute the program instructions to cause the system to:
receive patient data associated with a patient;
determine at least one arc, the arc corresponding to anatomical points of teeth of a tooth library;
determine, based on the at least one arc, a double helix, the double helix to be used for fitting the tooth library;
determine positions of the teeth of the tooth library on the double helix; and
optimize the teeth of the tooth library.

18. (canceled)

19. (canceled)

20. The system of claim 17, wherein determining the at least one arc comprises:
providing the patient data to an AI model, the AI model trained to identify the anatomical points of the teeth of the tooth library;
determining a centering arc;
determining a fitting arc; and
determining an aesthetic arc, and wherein determining the aesthetic arc comprises:
projecting one or more control points onto an image of the patient;
defining based at least in part on the one or more control points, an initial curve;
defining a final curve by modifying at least one control point of the one or more control points; and
determining locations of one or more anatomical points based at least in part on the final curve, the locations of the one or more anatomical points defining at least in part the aesthetic arc.

21. The system of claim 17, wherein determining the double helix comprises providing the at least one arc to an AI model configured to determine the double helix based at least in part on the at least one arc.

22. The system of claim 17, wherein optimizing the teeth of the tooth library comprises determining, using an AI model, a position, a rotation, or both of each tooth of the tooth library, the AI model configured to optimize functional and aesthetic positioning of the teeth.

23-32. (canceled)
